(19) United States
(12) Patent Application Publication
(10) Pub. No.: US 2002/0065225 A1
(43) Pub. Date: May 30, 2002

(54) Methods of determining altered NDPK functions and the diagnosis of cystic fibrosis

(76) Inventors: Richmond Muimo, Leeds (GB); Anil Mehta, Angus (GB)

Correspondence Address: Braman & Rogalskjy, LLP, PO Box 352, Canandaigua, NY, 14424-0352, US

(21) Appl. No.: 09/944,030

(22) Filed: Aug. 31, 2001

Related U.S. Application Data
(63) Continuation of application No. PCT/GB00/00736, filed on Mar. 2, 2000.
(60) Provisional application No. 60/122,426, filed on Mar. 2, 1999.

(51) Int. Cl. G01N 33/574; C07K 7/08; A61K 38/10

(52) U.S. Cl. 514/14; 435/7.23; 530/327

(57) ABSTRACT

A peptide of relative molecular mass less than 6500 comprising at least ten consecutive amino acid residues surrounding the phenylalanine (508), or at least ten consecutive residues including a portion of the region between residues (508 and 551), in the polypeptide sequence of human cystic fibrosis transmembrane regulator (CFTR), or a variant or precursor thereof. A peptide as defined above having between 12 and 50 amino acid residues. Methods of treating cystic fibrosis are also disclosed. A method of classifying a disease state associated with epithelial cell dysfunction in a patient is disclosed. The method includes obtaining a suitable epithelial cell sample from the patient and determining for one or more of the following whether the measured parameter is altered compared to a control epithelial cell, the measured parameters being: (i) nucleoside diphosphate kinase (NDPK) function, (ii) phosphorylation of annexin, (iii) phosphorylation of other membrane proteins, and (iv) ATPase activity.

The present invention relates to compounds which are believed to be useful in the treatment of cystic fibrosis (CF) and it also relates to various methods of screening patients in order to determine whether they have CF, and to methods of screening for drugs which may be useful in the treatment of CF.

CF is the most important autosomal recessive inherited cause of early death of young adults in the UK.

CF results from mutations in the cystic fibrosis transmembrane-conductance regulator protein (CFTR) which controls epithelial transport. The defects in this gene are many (over 500 described to date). In about 70% of cases the defect is very simple, being the loss of the phenylalanine residue at position 508 in the protein (ΔAF508 in CFTR). Having lost this amino acid, epithelial cells fail to function properly. Specifically, they fail to regulate the movement of ions across their membranes. This results in a very sticky lung mucus and failure to digest food properly. The mechanism is unknown.

CF is typically diagnosed by genetic methods. Other methodologies for analysing CF defects include physiological tests such as a sweat test, measuring nasal potential difference, and loading lung cells with chloride and measuring Cl⁻ exit via fluorescence. The latter two methods are generally considered to be experimental techniques.

A further technique which is sometimes used is screening at birth by measurements of the blood concentration of the enzyme, trypsin. This test is complementary to the gene test.

Most current efforts at developing treatment for CF are centred around replacing the gene; other attempts are centred on the physiological abnormalities found in CF and on the correction of the abnormal movement of ions. So far no patient has actually received any gene therapy as such outside a safety study.

Despite the cloning of the CFTR gene, the exact mechanism by which the genetic defect causes disease is not understood.

Nucleoside diphosphate kinase (NDPK) synthesises various nucleoside triphosphates (N₂TP) from their corresponding nucleoside diphosphates (N₂DP) such that N₁TP+N₂DP→N₁DP+N₂TP, where N₁TP is the nucleoside triphosphate donating the γ-phosphate.

NDPK is essential for cell secretion and differentiation and is the sole cellular source of UTP. UTP is a (non-CFTR) chloride secretogogue in airway epithelia. It has been shown previously [sodium ⇓], [potassium ↑], and [chloride ↑] differentially regulate NDPK activity at physiologically relevant concentrations (Treharne et al (1994) Am. J. Physiol. (Lung Cell. Mol. Physiol.) 267, L592-L601; Marshall et al (1998) J. Physiol. 507, 3-4; and Muimo et al (1999) Am. J. Physiol. Cell. 276, C109-119). NDPK is also a transcription factor formerly known as PUF. The mode of action of NDPK in the regulation of transcription is not understood. Muimo et al (1997) Am. J. Respir. Cell Mol. Biol. 17, 1-9 describes NDPK and Cl⁻ sensitive protein phosphorylation in apical membranes from ovine airway epithelium. Anciaux et al (1997) FEBS Lett. 400, 75-79 describes the inhibition of NDPK by cyclic AMP analogues. Tsao et al (1998) Am. J. Respir. Cell Mol. Biol. 18, 120-128 describes degradation of Annexin I in bronchoalveolar lavage fluid from patients with CF.

In our investigations, described in more detail in the Examples, we have found that the defective mucus secretion, excessive lung inflammation and disordered regulation of ion channels may be explained in a coherent manner by disordered function of a histidine-based signalling system involving the enzyme nucleoside diphosphate kinase (NDPK) which we have found interacts with a peptide corresponding to wild-type CFTR but does not interact (or does not interact in the same way) with mutant CFTR, in particular ΔF508 CFTR.

In particular, we have found amongst other things that (1) NDPK is defective (but present) in transgenic mice not expressing CFTR but that it is functionally restored following transfection of the mice with a plasmid vector containing a CFTR insert; (2) phosphorylated NDPK binds wild-type (but not disease-associated peptides) corresponding to the CFTR region carrying the common disease mutation, ΔF508; (3) NDPK phosphorylation is defective in plasma membranes from airway epithelia of cam/cam (-/-) mice (which express no CFTR); (4) membranes from Cftr⁻ mice are unable to augment the synthesis of trinucleotides from corresponding dinucleotides; (5) NDPK can be affinity purified with peptides derived from wild-type ovine CFTR containing the F508 residue; and (6) histidine phosphorylation of annexin I (Anx I) is dependent on CFTR since Anx I phosphorylation is attenuated in CFTR-null tracheal epithelium but restored when the cells are transfected with a CFTR expression plasmid.

The present invention makes use of these observations by providing compounds which may be useful in restoring the proper function of NDPK in cells which are defective for CFTR; by providing methods to evaluate the efficacy of potential drugs and gene therapies in the treatment of CF; and by providing a means of determining whether a patient truly has CF or not since in around 10% or so of cases of suspected CF, genetic and physiological tests (such as the sweat test mentioned above) do not give a clear picture of the disease.

A first aspect of the invention provides a peptide of relative molecular mass less than 6500 comprising at least ten consecutive amino acid residues surrounding the phenylalanine 508, or at least ten consecutive residues including a portion of the region between residues 508 and 551, in the polypeptide sequence of human cystic fibrosis transmembrane regulator (CFTR), or a variant or precursor thereof.

Preferably, the peptide has a relative molecular mass less than 5000; more preferably less than 4000; still more preferably less than 3000; and it may have a relative molecular mass less than 2000.

The amino acid sequence of human CFTR is described in Kerem et al (1989) Science 245, 1073-1080 and the amino acid sequence of the mouse CFTR is described in Ratcliffe et al (1993) Nature Genetics 4, 35-41. The amino acid sequence immediately surrounding the phenylalanine 508 reside (F508) in human CFTR is KENIIFGVSYDEYR and in sheep is KDNIIFGVSYDEYR (the F508 residue is in bold and is underlined). The mouse and sheep peptides can be considered to be variants of a human peptide as described below.

Typically, the peptide has between 12 and 50 amino acid residues. Preferably, the peptide has between 12 and 30 amino acid residues; more preferably the peptide has between 12 and 20 amino acid residues. A particularly preferred peptide which binds to NDPK is the peptide which has the sequence KENIIFGVSYDEYR.

In one preferred embodiment the peptide has the sequence as found from residues 508 to 551 in human CTFR, or a variant or precursor thereof.

The peptides of the invention are believed to be useful in treating CF and chronic bronchitis and its variants, including chronic sputum producing disorders. Without being bound by any theory as to why the peptides are useful, at least some of the peptides as disclosed (for example, some of those in the Examples) are able to bind NDPK and it is believed that in so doing they may restore properties to NDPK which are lost or reduced in sufferers of CF in which the CFTR polypeptide is defective, for example by virtue of the lack of phenylalanine 508 (ie the Δ508 mutation) or sufferers of chronic bronchitis and its variants.

Thus, restoring a binding site of CFTR via a peptide may restore phosphorylation in vivo thus obviating the need for a full length protein.

It will be appreciated that the peptide of the invention may have at least ten amino acid residues surrounding the F508 of human CFTR; however in one embodiment of the invention the peptide is a variant in which between one and four of the amino acids in the at least ten surrounding the F508 are replaced by other amino acid residues. Preferably, the replacement amino acids are conservative substitutions of the amino acids found in the natural protein. By “conservative substitutions” we include substitutions in which one hydrophobic amino acid residue is replaced by another hydrophobic ammo acid residue; also included is the substitution of one negatively charged amino acid residue with another negatively charged amino acid residue, and the replacement of one positively charged amino acid residue with another positively charged amino acid residue.

Thus “conservative substitutions” include substitutions within the groups Gly, Ala; Val, Ile, Leu; Asp, Glu; Asn, Gin; Ser, Thr; Lys, Arg; and Phe, Tyr.

Similarly, variants of a peptide which has at least ten consecutive residues and includes a portion of the regions between residues 508 and 551 include peptides in which conservative substitutions have been made.

It is preferred if the amino acid residues that include and surround F508 in the peptide of the invention are the same as the amino acid residues which are present in the natural wild type polypeptide at equivalent positions. If the amino acid residues are different to those in the natural wild-type polypeptide it is preferred if the residue at the position equivalent to F508 is a hydrophobic residue. Similarly, it is preferred if the amino acid residue at the position equivalent to either position 506 or 507 are hydrophobic residues (they are both isoleucine (I) in the natural wild type polypeptide). In the case of variants it is preferred that the peptide is still able to bind to NDPK. Whether or not a peptide can bind to NDPK can be determined using the affinity chromatography methods described in the Examples. Preferably, the peptides of the invention are ones which bind to NDPK in a substantially similar way to the peptide which has the sequence KENIIFGVSYDEYR.

Peptides may be synthesised by the Fmoc-polyamide mode of solid-phase peptide synthesis as disclosed by Lu et al (1981) J. Org. Chem. 46, 3433 and references therein. Temporary N-amino group protection is afforded by the 9-fluorenylmethyloxycarbonyl (Fmoc) group. Repetitive cleavage of this highly base-labile protecting group is effected using 20% piperidine in N,N-dimethylformamide. Side-chain functionalities may be protected, as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine). Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalising agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N,N-dicyclohexyl-carbodiimide-1-hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used are ethanedithiol, phenol, anisole and water, the exact choice depending on the constituent amino acids of the peptide being synthesised. Trifluoroacetic acid is removed by evaporation in vacuo, with subsequent trituration with diethyl ether affording the crude peptide. Any scavengers present are removed by a simple extraction procedure which on lyophilisation of the aqueous phase affords the crude peptide free of scavengers. Reagents for peptide synthesis are generally available from Calbiochem-Novabiochen (UK) Ltd, Nottingham NG7 2QJ, UK. Purification may be effected by any one, or a combination of, techniques such as size exclusion chromatography, ion-exchange chromatography and (principally) reverse-phase high performance liquid chromatography. Analysis of peptides may be carried out using thin layer chromatography, reverse-phase high performance liquid chromatography, amino-acid analysis after acid hydrolysis and by fast atom bombardment (FAB) mass spectrometric analysis.

By “peptide” we include not only molecules in which amino acid residues are joined by peptide (—CO—NH—) linkages but also molecules in which the peptide bond is reversed. Such retro-inverso peptidomimetics may be made using methods known in the art, for example such as those described in Méziere et al (1997) J. Immunol. 159, 3230-3237, incorporated herein by reference. This approach involves making pseudopeptides containing changes involving the backbone, and not the orientation of side chains. Méziere et al (1997) show that, at least for MHC class II and T helper cell responses, these types of pseudopeptides are useful mimics of peptides. Retro-inverso peptides, which contain NH—CO bonds instead of CO—NH peptide bonds, are much more resistant to proteolysis.

Similarly, the peptide bond may be dispensed with altogether provided that an appropriate linker moiety which retains the spacing between the Cα atoms of the amino acid residues is used; it is particularly preferred if the linker moiety has substantially the same charge distribution and substantially the same planarity of a peptide bond.

It will be appreciated that the peptide may conveniently be blocked at its N- or C-terminus so as to help reduce susceptibility to exoproteolytic digestion. Peptide mimics which are resistant to proteolytic digestion are preferred. Resistance to proteases which are found in the lungs or: gut of CF sufferers is particularly preferred.

A precursor of a peptide of the invention is a molecule which , following administration to the patient, is converted into a peptide as defined. The precursor is, therefore, a form of propeptide or prodrug. Suitably, the precursor is one which is acted upon by a protease present within the body of the patient, for example a serum protease, such that a larger peptide is converted into a peptide as defined. Precursors can readily be designed and synthesised by reference to known cleavage sites of proteases.

Myristylated peptides or peptides with a steroid head group are suitable precursors. Enzymes are present in the body which are able to remove the head groups and lipid tails and release active material into the cell.

A second aspect of the invention provides a peptide according to the first aspect of the invention and a lipid-solubilising moiety.

For the avoidance of doubt, the relative molecular mass less than 6500 is the relative molecular mass of the peptide portion alone of any molecule further comprising a lipid-solubilising moiety, and does not include the relative molecular mass of the lipid-solubilising moiety. Similarly; it refers to the relative molecular mass of the peptide or variant and not to a precursor thereof.

The lipid solubilising moiety may be any moiety which, when attached to the peptide, permits the peptide to partition into a lipid phase, for example into the cell membrane. Preferably, the lipid-solubilising moiety is one which allows the peptide to segregate preferentially into the inner leaflet of the cell membrane such that the peptide faces the cytosol. The lipid-solubilising moiety may be a phospholipid. Phospholipids that preferentially segregate to the inner leaflet of the cell membrane include serine- and ethanolamine-containing phospholipids.

The lipid-solubilising moiety may also be a suitable fatty acid or other lipid molecule such as a cholesterol. Suitable such molecules include myristyl and palmityl groups, and cholesterol. Acidic phospholipids may also be useful since they may bind to annexin.

It is particularly preferred if the peptide of the invention fierier comprising a lipid-solubilising moiety is one which can restore a macromolecular assembly in a cell which contains NDPK and an annexin, particularly a lung epithelial cell that has a CFTR Δ508 mutation.

Lipids may be attached to peptides using acetyl transferases and myristyl transferases, or they may be attached using chemical methods known in the art.

A third aspect of the invention provides a peptide according to the first or second aspects of the invention packaged and presented for use in medicine.

As noted the peptides of the invention are believed to be useful in the treatment of CF and chronic bronchitis and its variants, including chronic sputum producing disorders. It is particularly preferred if the peptides are prepared as a pharmaceutical composition or formulation suitable for administration to a patient. Thus, a fourth aspect of the invention provides a pharmaceutical formulation comprising a peptide according to the first or second aspects of the invention together with one or more pharmaceutically acceptable carriers therefor and optionally one or more other therapeutic ingredients. The carrier(s) must be ‘acceptable’ in the sense of being compatible with the other ingredients of the formulation and not deleterious to the recipient thereof. Typically, carriers for injection, and the final formulation, are sterile and pyrogen free.

The formulations include those suitable for oral or nasal (particularly inhaled), parenteral (including subcutaneous, transdermal, intradermal, intramuscular and intravenous and rectal) administration, although the most suitable route may depend upon for example the condition and disorder of the recipient. The formulations may conveniently be presented in unit dosage form and may be prepared by any of the methods well known in the art of pharmacy. All methods include the step of bringing into association a compound of the present invention as herein defined or a pharmacologically acceptable salt or solvate thereof (“active ingredient”) with the carrier which constitutes one or more accessory ingredients.

Formulations of the present invention suitable for oral admiration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil. liquid emulsion. The active ingredient may also be presented as a bolus, electuary or paste. Formulations for inhalation may be presented in any of is the ways known to be effective eg metered dose inhalers.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter or polyethylene glycol.

A fifth aspect of the invention provides a method of treating CF or a chronic sputum producing disorder the method comprising administering to the patient an effective amount of a peptide according to the first or second aspect of the invention. By an “effective amount” we mean an amount which is useful in preventing or ameliorating or eliminating the symptoms of CF or the chronic sputum producing disorder. Chronic sputum producing disorders such as chronic bronchitis and its variants are readily diagnosed by a physician. Whether or not an individual has CF may be determined genetically as is well known in the art or may be determined by the methods described below. Similarly, the efficacy of the peptides of the invention may be determined using methods described below.

It will be appreciated that it is particularly useful to administer the peptide of the first or second aspect of the invention to the site where the symptoms of CF are manifested. In particular, it is preferred if the peptides are administered to the lungs, for example using a nebuliser, or if they are administered to the gut by any convenient method. Similarly, in relation to treating chronic sputum producing disorders, the peptides of the invention are advantageously delivered to the lungs.

The peptides may be useful in the treatment of other diseases involving NDPK and annexin. NDPK is believed to be involved in certain tumours. Also, annexin may be involved in regulating the secretory capacity of the gut, pancreas and neutrophils. The entry of Salmonella tphimurium is controlled by CFTR.

The peptides of the invention may be delivered to the site they are required by different methods.

(1) Delivery to lungs using neutrophils. Neutrophils escape from the blood into the lung and do so in an excessive manner in CF. Patient's neutrophils may be removed from the blood, modified to insert the peptide and reinjected to deliver the therapeutic peptide to the lung.

(2) Delivery via a liposome. Typically cationic lipids are used, such as those developed by Genzyme.

(3) Selective targeting to duodenum, jejenum using gut epithelial cell markers eg caveolin receptor, E. coli pilin protein, sugar polymers which are gut section specific. Gut is involved in CF and oral delivery of peptides which are resistant to acid attack are useful.

(4) Via reticuloendothelial system.

The patient is preferably a human patient. It will be appreciated that it is preferred that the peptides or variants administered to a human patient are ones which are based on the sequence of human CFTR, although they may, as discussed above, be based on the sequence of other mammalian CFTR.

A sixth aspect of the invention provides the use of a peptide according to the first or second aspect of the invention in the manufacture of a medicament for treating CF or chronic bronchitis and its variants including chronic sputum producing disorders.

Although CF is mostly diagnosed in a patient using genetic methods, or using the sweat test or other physiological methods, these tests do not always give a firm diagnosis. For example, although around 70% of CF mutant alleles have the ΔF508 mutation, this means that only around 50% of CF sufferers are homozygous for this mutation. Thus, other mutations must be present, and although many such other mutations have been found, around 10% of CF cases (or cases which are believed to be CF) cannot be diagnosed using genetic methods. Thus, other methods of diagnosing CF in a patient are desirable.

The ability to be able to better determine whether an individual has CF or not is very important for several reasons. Firstly, if an individual is incorrectly diagnosed as not having CF when the individual does, in fact, have CF, he or she may not be given appropriate treatment. Since it is particularly important that treatment is initiated at an early age in order to give the maximum chance of increasing life expectancy, a proper diagnosis is very desirable. Similarly if an individual is incorrectly diagnosed as having CF when the individual does not, in fact, have CF, he or she may be treated unnecessarily. Again this is very important since treatment is often in the form of intensive physiotherapy given many times a day by the parents or other carers of the child over many years; typically, the treatment takes two hours per day including medication. Thus, the ability to eliminate or reduce the chances of a child unnecessarily being treated in this way would reduce emotional and physical stress in the child and his or her carers, and would eliminate or reduce the chances of health service resources being incorrectly used.

In some cases, an individual may have been classified genetically as having CF (because they have a mutation in the CFTR gene which, ostensibly, is one which may be associated with CF) but they only have very mild symptoms. Nevertheless, because they have been classified genetically as having CF they may be treated (inappropriately) in the same way as, for example, an individual who has much more severe CF (eg an individual who is homozygous for ΔF508 in CFTR).

Thus, in some cases, it would be useful to classify an individual as one who although genetically has CF, is not one who physiologically has CF to the extent that the individual requires treatment, or at least the same intensive treatment as an individual with a severe form of CF.

Similarly, it is useful to determine whether a CF patient is responding to a particular treatment.

A seventh aspect of the invention provides a method of determining whether a patient has, or is responding to treatment for, CF, the method comprising the steps of (1) obtaining a suitable epithelial cell sample from the patient, (2) determining whether nucleoside diphosphate kinase (NDPK) function or state is altered compared to its function or state in a control epithelial cell.

Conveniently, alteration in NDPK function may be assessed by determining whether phosphorylation of NDPK, is altered. The phosphorylation of NDPK may be measured from the counts per minute incorporated into NDPK from γ³²P ATP or GTP per mg of membrane protein. Typically, the amount of phosphate incorporated from ATP or GTP is in the fmoles of phosphate/mg membrane protein.

Phosphorylation of epithelial membrane components, including that of NDPK, have been describe in Treharne et at (1994) Am. J. Physiol. (Lung Cell. Mol. Physiol.) 267, L592-L601; Muimo et al (1998) Am. J. Respir. Cell. Mol. Biol. 18, 270-278; and Marshall et al (1999) Am. J. Physiol. (Cell Physiol.) 276, C109-119, incorporated herein by reference. Briefly, after characterisation of epithelial membrane fractions (plasma, apical, endoplasmic reticulum, cytosol), the time course of histidine phosphorylation may, if appropriate, be determined under different ionic conditions (Δ: [Na⁺], [K⁺], [Cl⁻], pH). Phosphorylation is initiated with either γy[³²P]ATP or γ[³²P]GTP in the presence of di/trinucleotide combinations.

In one embodiment, nucleotide synthetic activity can be used as an assessment of NDPK function.

For assessment of NDPK function, a suitable amount of the phosphorylation reaction mixture is removed and the reaction terminated with EDTA. The nucleotide synthetic activity can be assayed by TLC or by HPLC. When TLC is used, the nucleotides may, for example, be detected using a Canberra-Packard Instant Imager.

The phosphorylation state of NDPK is regulated in normal cells by many factors. In the CF cell, NDPK is dephosphorylated.

Conveniently, the alteration in NDPK function may be assessed by measuring nucleotide triphosphate production from a given nucleoside diphosphate. The loss of NTP production in CF is described in the examples. Nucleotide production may be measured as described in the Examples using thin layer chromatography which separates the radiolabelled nucleotides. Alternatively, HPLC methods may be used. Tritiated nucleotide standards may be useful in the measurement of the production of UTP, ATP or GTP. Typically, the fmole amounts of trinucleotides synthesised from dinucleotides per minute per mg membrane protein are measured.

The production levels of NTP are almost zero in a CF cell.

For example, apically enriched membranes from airway epithelia which contain NDPK may be isolated. GTP and ATP production may be assessed using (respectively) either 16 nM γ[³²P]ATP+500 nM GDP or 16 nM γ[³²P]GTP+500 nM ADP in the airway epithelial membranes. Typically, 2.5 μg of membrane protein is incubated for up to 5 min at 37° C. as described in Treharne et al (1994) Am. J. Physiol. 267, L592-L601 and Muimo et al (1997) Am. J. Lung Cell Mol. Biol. 17, 1-9. N₂TP production may be measured by TLC and quantitated by electronic autoradiography. Using this methodology, nucleotide synthesis was found to be deficient in airway membranes from null CFTR mice.

An eighth aspect of the invention provides a method of determining whether a patient has, or is responding to treatment for, CF the method comprising the steps of (1) obtaining a suitable epithelial cell sample from the patient, (2) determining whether histidine phosphorylation of annexin is altered compared to its phosphorylation in a control epithelial cell.

Annexins are descried in The Annexins, Moss, SE (ed) Portland Press, UK, 1992 and their participation in protein phosphorylation is described in Rothhut (1997) Cell Mol. Life Sci. 53, 522-526, both of which are incorporated herein by reference.

Histidine phosphorylation of annexin may be measured using any suitable method including, for example, SDS-PAGE and autoradiography (following the use of a radiolabelled phosphate donor), and/or immunoprecipitation. A suitable method of assessing histidine phosphorylation of annexin I is described in detail in Example 2.

In a normal cell annexins I and II (Anx I and II) are phosphorylated whereas in a CF cell they are almost completely dephosphorylated.

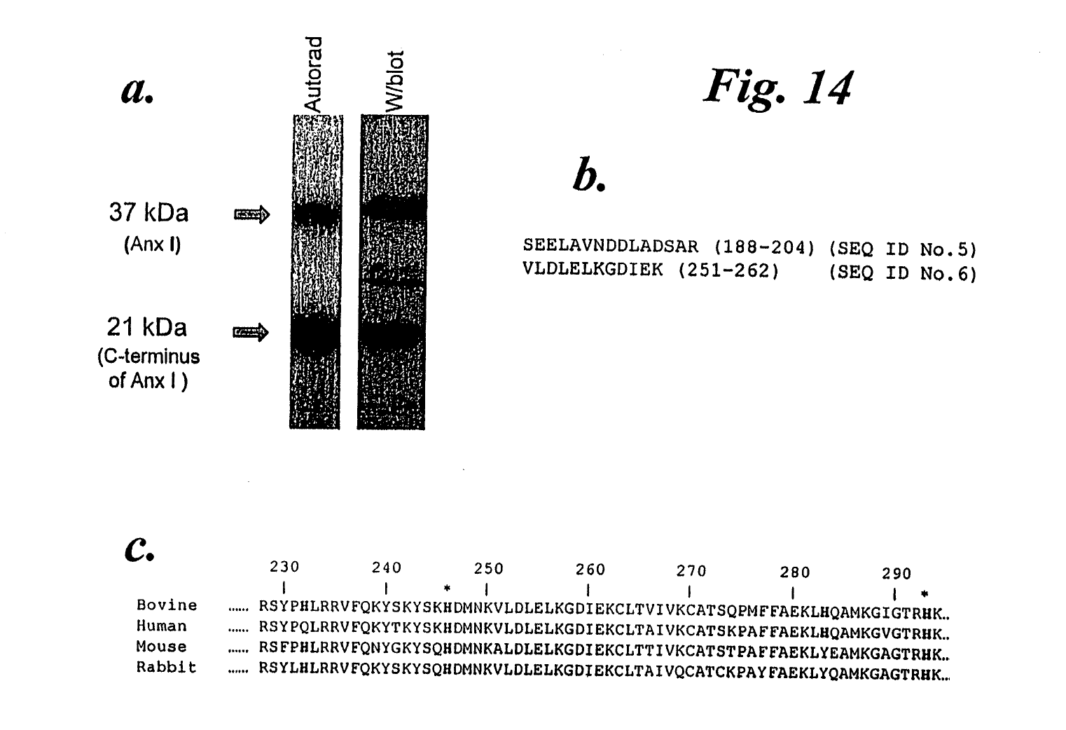

As is described in the Examples, annexin appears to be histidine phosphorylated at histidine 246 or histidine 293 (see FIG. 14). Thus, it is preferred if the measurement of histidine phosphorylation of annexin is measurement of the phosphorylation of histidine 246 or histidine 293. Measurement of phosphorylation of these specific histidine residues may be carried out by any suitable means. However, in a preferred embodiment of the invention an antibody which selectively binds to the specifically phosphorylated annexin but which does not bind to annexin not phosphorylated at the specific histidine residues is used.

A ninth aspect of the invention provides a method of classifying a disease state associated with epithelial cell dysfunction in a patient, the method comprising (1) obtaining a suitable epithelial cell sample from the patient and (2) determining for one or more of the following whether the measured parameter is altered compared to a control epithelial cell the measured parameters being: (i) nucleoside diphosphate kinase (NDPK) function, (ii) phosphorylation of annexin, (iii) phosphorylation of other membrane proteins, and (iv) ATPase activity.

Typically, membrane fractions of the epithelial cells are made and the activities measured therein. Suitably, plasma membranes are prepared and the activities are measured therein. Preferably, an apically-enriched fraction is used; however, any suitable fraction may be used including a fraction which contains basolateral membrane.

Typically, annexin is phosphorylated on a histidine residue and it is this that is measured. As noted above, typically histidine phosphorylation is on histidine 246 or histidine 293 and it is this histidine phosphorylation that is measured. We have observed reduced phosphohistidine content of the plasma membrane from epithelial cells from CF nasal samples compared to control samples. Histidine phosphorylation of annexin may be assessed as described above and as described in more detail in Example 2.

Additionally, it may be useful to measure the ion sensitivity. This is the state where sodium ion concentrations in excess of 10 mM inhibit NDPK but only when ATP is present; this does not occur when GTP is a substrate. Chloride ions lead to increased phosphorylation of NDPK when the concentration rises above 40 mM.

An ATPase activity is found in normal control samples whereas in CF samples this is not found to be present. ATPase can be measured by determining a reduction in the amount of ATP in the sample against time, for example using radiolabelled ATP. Without being bound by any theory, the reduction in ATP may be due to the γ-phosphate being transferred from the ATP to water (hydrolysis), to annexin II or to NDPK.

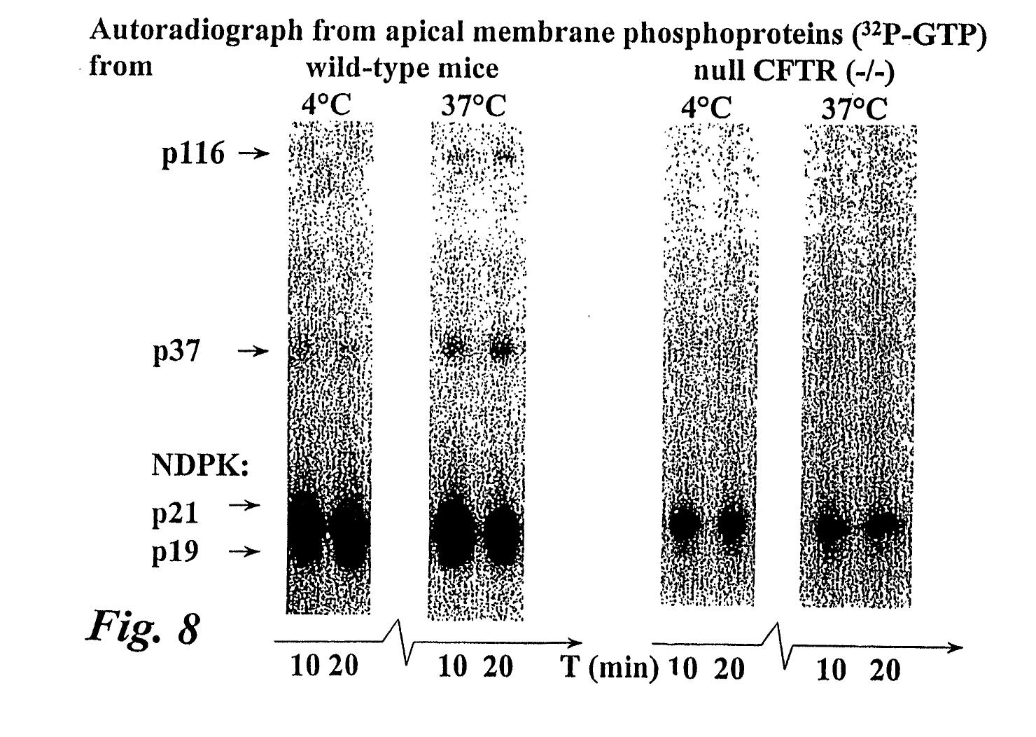

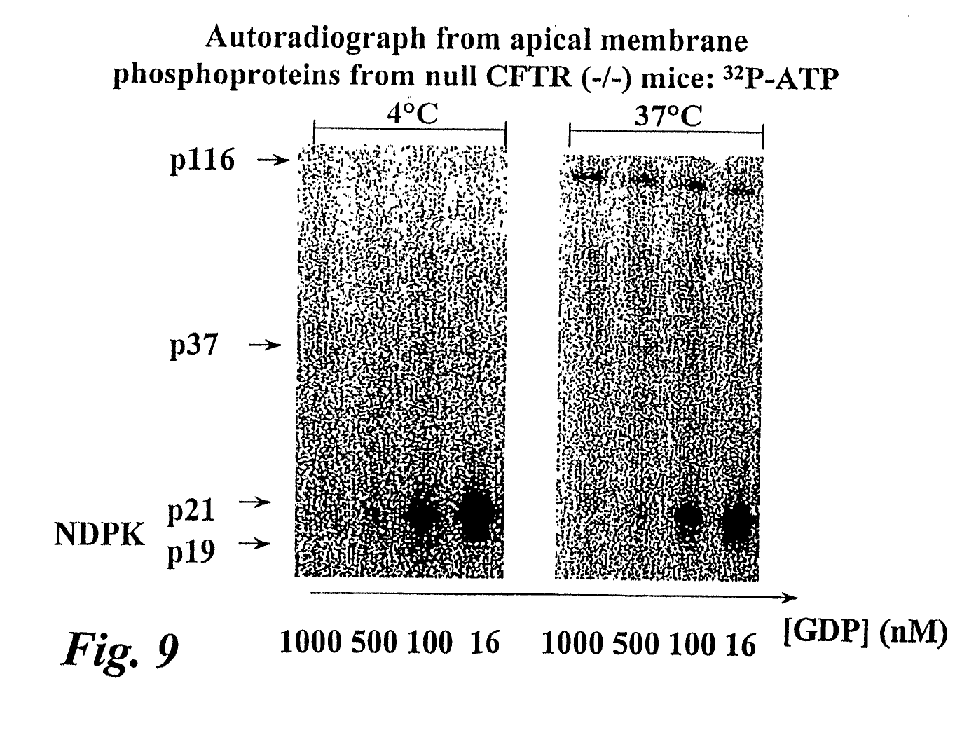

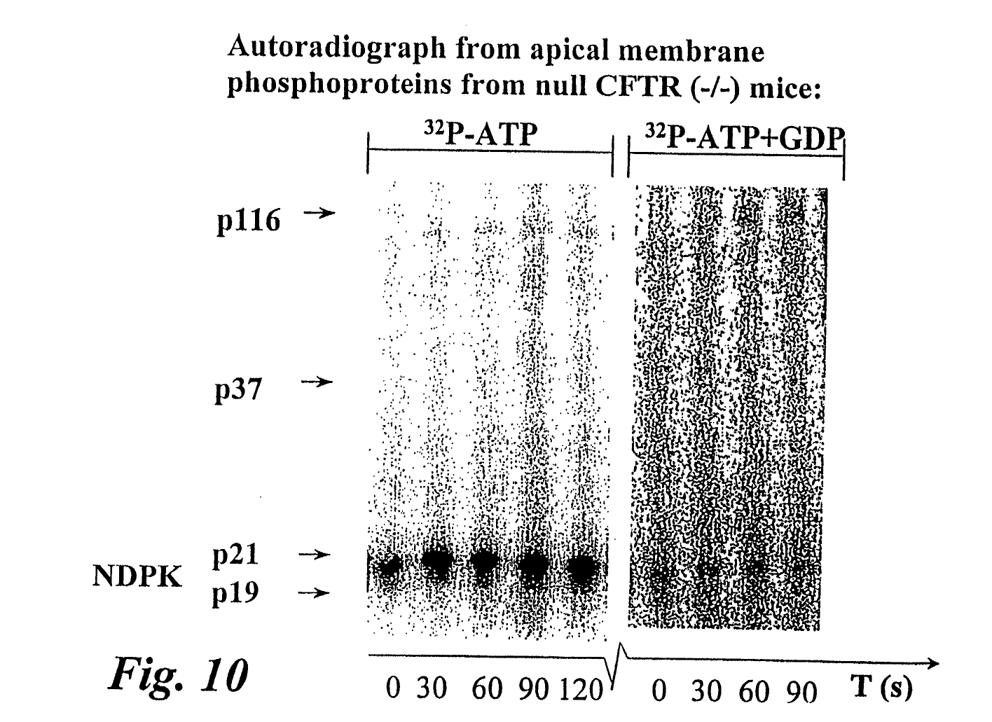

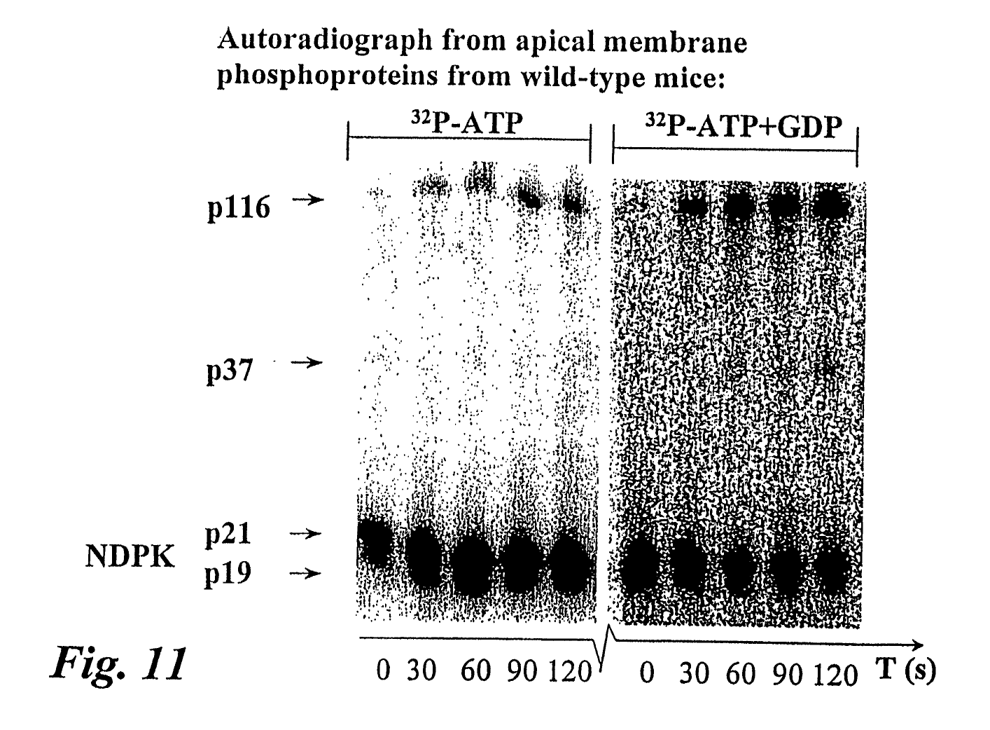

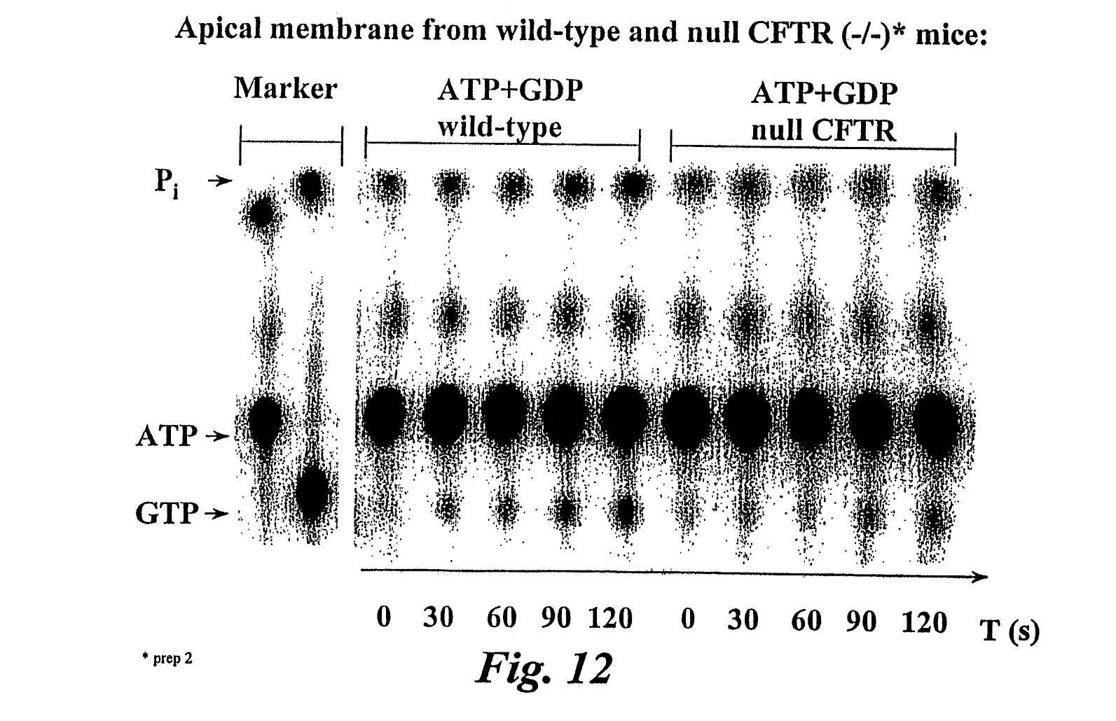

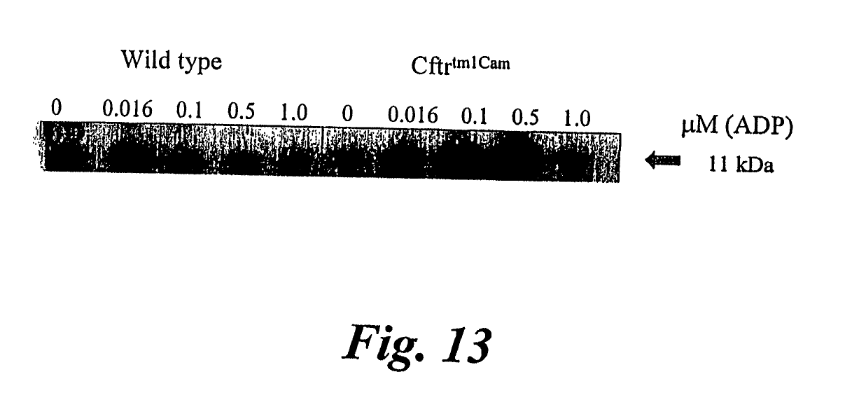

Phosphorylation of other membrane proteins may be measured using autoradiography. The phosphorylation state of at least two other membrane proteins is altered in CF cells compared to control cells. These we have designated p11 and p116 based on approximate molecular weights on SDS-PAGE of 11 kDal and 116 kDal. The protein p11 is hyperphosphorylated in cystic fibrosis. Phosphorylation of p116 is enhanced when UDP is present with ATP. With the exception of p11, the other membrane protein show a lower phosphorylation in CF compared to normal cells. (See FIGS. 8 to 13.) The ion sensitivity of phosphorylation in normal cells is that of sodium inhibition and chloride activation and potassium activation. This does not occur in CF cells. Phosphorylation of p11 and p116 may be measured in essentially the same way as that of annexin.

Preferably, at least two of the parameters are measured in the sample in the patient and compared to the control sample; more preferably at least three of the parameters are measured in the sample in the patient and compared to the control sample.

Preferably, parameters (i) and (ii) are measured in the sample in the patient and compared to the control sample.

More preferably, each of parameters (i), (ii) and (iii) are measured in the sample from the patient and compared to the control sample.

Still more preferably, all of the parameters (i) to (iv) are measured in the sample from the patient and compared to the control sample.

The methods of the seventh, eighth and ninth aspect of the invention may conveniently be used to determine the effectiveness of a proposed treatment for CF. As has been noted, treatment of CF by gene therapy (for example, the introduction into affected epithelial cells of a cDNA encoding natural wild-type CFTR in an appropriate vector) has been proposed and carried out in trials; similarly, the method of the fifth aspect of the invention provides a method of treating CF. It is important to be able to assess the effectiveness of any proposed or actual treatment regimes including not only gene therapy regimes but also drug-based treatment regimes. Thus, in these particular embodiments of these seventh, eighth and ninth aspects of the invention, the appropriate epithelial cell samples are isolated from the patient before treatment begins and the given parameters measured in order to establish a “base line” for each of the parameters, and further samples of appropriate epithelial cells are taken at appropriate intervals during treatment and the given parameters measured in order to establish what effect the treatment is having on the patient. Appropriate intervals may be monthly.

The treatment is considered to be having a desirable effect if for the parameters measured they become more like the equivalent parameters in a known normal (ie non-CF) cell sample and less like the equivalent parameters in a known CF cell sample.

The method of the ninth aspect of the invention may be used to confirm that an individual who genetically has CF manifests biochemical changes that indicate a severity of CF which warrants treatment. It may also be used, to indicate that an individual does not have a severity of CF which warrants treatment, despite genetically having CF. It is particularly preferred if the status of NDPK function, annexin phosphorylation, p11 phosphorylation and p116 phosphorylation are measured. The method may also be used to indicate that a patient has CF despite no mutation being detectable in the CFTR gene or no mutation being present which is a mutation determined previously to be associated with CF. Typically, annexin phosphorylation is histidine phosphorylation and it is preferred to measure phosphorylation of histidine 246 or histidine 293 of annexin.

Successful treatment of a patient may be indicated by dephosphorylation of p11, and the addition of phosphate in NDPK, annexin, p11 or p116.

The methods of the invention may also be used to help classify or identify unexplained lung diseases such as chronic bronchitis.

Thus, the method of the ninth aspect of the invention allows for patients with an epithelial cell dysfunction, such as CF, to be classified more precisely than by the current genetic methods.

The sample of epithelial cell may be prepared into any fractions and fractions may be used in which any one or more of the parameters described, such as NDPK function, annexin phosphorylation (preferably histidine phosphorylation of residues 246 or 293), changes in phosphorylation of membrane protein, and ATPase activity may be measured. Apart from changes in phosphorylation of membrane protein, the parameters described may be measured in fractions including cytosolic fractions, endoplasmic reticulum fractions and plasma membrane fractions, including both apically-enriched fractions and basolateral fractions. It is preferred if plasma membrane fractions-are used; it is further preferred if apically-enriched fractions of plasma membrane are used.

Typically, in screening a patient according to the ninth aspect of the invention, an epithelial cell biopsy is taken (for example, a lung biopsy) and epithelial cell membranes are prepared.

Radiolabelled ATP/GTP (γ³²P ATP and γ³²P GTP) is used and phosphorylation of NDPK, annexins, or other airway epithelium membrane proteins is measured following the addition of the radiolabel to the membrane, using electronic autoradiography.

With respect to the seventh, eighth and ninth aspects of the invention, and as the case may be, the epithelial cell sample tested may be any suitable cell sample in which dysfunction may be found, particularly dysfunction due to CF. Typically, the epithelial cell sample is a lung cell sample, or a nasal epithelial cell sample, or a gut epithelial cell sample or liver biopsy. Preferably, because of the ease with which it can be obtained, the epithelial cell sample is a nasal epithelial cell sample. Suitable nasal epithelial cell samples may be obtained by scraping or brushing the lining of the nose. A typical assay requires two brush strokes per nostril. Methods for separating appropriate membrane fractions from post nuclear supernatants using 2 ml sucrose gradients and a TST 55.5 rotor have been described previously (Treharne et al (1994) Am. J. Physiol. (Lung Cell. Mol. Physiol.), 267, L592-L601; incorporated herein by reference). Brushings may be stored, if necessary. Collection of nasal epithelial cells, their fractionation and determination of phosphorylation of membrane proteins is described in Example 3.

The control epithelial cell sample may be any suitable control sample. Suitable control samples include, for example, a known normal (ie non-CF) cell sample, or a known CF cell sample. Both types of control samples may advantageously be used. It will be appreciated that the control sample and the test sample may usefully be matched such that, for example, both the test and control cell samples are both from the same tissue type (eg both nasal samples), or both samples are otherwise genetically similar (eg from siblings), or age matched. Familial variability of NDPK and annexin may be studied in parents of affected cases.

Because CF patients are susceptible to infections, particularly bacterial infections of the lungs it is particularly preferred if the patient is treated with an appropriate course of antibiotics before the sample of epithelial cells (particularly nasal or lung epithelial cells) is isolated for measurement of the given parameters (and indeed the control samples are preferably obtained from an individual who has been treated with antibiotics such that the epithelial cells are free of a bacterial infection, or are otherwise free of such an infection).

The comparison of the test sample with that of a control sample may be measured directly, for example by carrying out the determination of the test parameters in parallel in test samples (derived from the patient) and in control samples (derived from suitable controls as discussed above). However, comparison of the test sample with control sample may be carried out indirectly in the sense that parameters to be measured for the control sample may have been measured historically and tabulated in a look-up table or the like so that it is only the parameters of the test sample that are measured when undertaking the determination. Control samples are suitably obtained from an individual who has been certified as having normal noses by a specialist ear, nose and throat (ENT) surgeon. The control samples may also be genotyped.

For each of the parameters eg NDPK function, histidine phosphorylation of annexin, phosphorylation of other membrane proteins, ion sensitivity, ATPase activity, loss of phosphorylation or activity indicates an alteration which is indicative of a disease or abnormal state, with the exception of phosphorylation of p11 where hyperphosphorylation is observed in CF samples compared to normal control.

It will be appreciated that in respect of the methods of the seventh, eighth and ninth aspects of the invention it is also particularly useful to determine the CFTR genotype of the patient, which can be done using methods well known in the art. The combination of CFTR genotype determination and determination of at least some of the parameters as set out in the methods of the seventh, eighth and ninth aspects of the invention may be particularly useful in aiding the physician and helping determine an appropriate treatment for the patient, if any.

A tenth aspect of the invention provides a method of identifying a compound useful in treating cystic fibrosis or which may aid the identification of a compound useful in treating cystic fibrosis the method comprising identifying a compound which modulates or restores nucleoside diphosphate kinase activity.

Typically, the compound is one which modulates the phosphorylation of NDPK.

Compounds which increase activity are particularly preferred.

The compound may be one which modulates the production of nucleoside triphosphates from a given nucleoside by NDPK.

NDPK is believed to phosphorylate annexin II and not annexin I. Identification of compounds which modulate the phosphorylation of annexin II by NDPK are believed to be useful.

NDPK activity may be measured by any suitable method. Suitable methods include those that lead to the production of radioactively or fluorescently labelled nucleoside triphosphates which can be measured using methods well known in the art. Typically, the conversion of GDP to GTP may be measured upon the addition of ATP and GDP; alternatively the conversion of ADP to ATP may be measured upon the addition of GTP and ADP. Other suitable nucleoside phosphates may be used such as UDP/UTP.

An eleventh aspect of the invention provides a method of identifying a compound, useful in treating cystic fibrosis or which may aid the identification of a compound useful in treating cystic fibrosis the method comprising, identifying a compound which modulates histidine phosphorylation of annexin. Histidine phosphorylation of annexin may be measured by any suitable method, such as by autoradiography and/or immunoprecipitation. Preferably, the compound is one which increases histidine phosphorylation of annexin. Preferably,. histidine phosphorylation of annexin is at residue 246 or 293.

A twelfth aspect of the invention provides a method of identifying a compound useful in treating cystic fibrosis or which may aid the identification of a compound useful in treating cystic fibrosis the method comprising identifying a compound which modulates the interaction between any of cystic fibrosis transmembrane conductance regulator protein (CFTR), nucleoside diphosphate kinase (NDPK) and annexin. The interaction may be assessed by identifying compounds which are able to restore the activity of NDPK and the phosphorylation state of annexin from that typical of a CF epithelial cell to that typical of a normal epithelial cell.

A further aspect of the invention provides a method of identifying a compound useful in treating cystic fibrosis or which may aid identification of a compound useful in treating cystic fibrosis the method comprising identifying a compound which substantially changes one or more of the following parameters from the state found in a cystic fibrosis epithelial cell to the state found in a normal cell, namely (i) nucleoside diphosphate kinase (NDPK) function, (ii) phosphorylation of annexin, (iii) phosphorylation of other membrane proteins such as p11 or p116, and (iv) ATPase activity.

The measurement of these parameters has been described above, and suitable assay formats may readily be devised. Preferably, phosphorylation of annexin is histidine phosphorylation at residues 246 or 293.

The methods may be carried out in any suitable in vivo or in vitro format.

The compound may be a drug-like compound or lead compound for the development of a drug-like compound for each of the above methods of identifying a compound. It will be appreciated that the said methods may be useful as screening assays in the development of pharmaceutical compounds or drugs, as well known to those skilled in the art.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons and which may be water-soluble. A drug-like compound may additionally exhibit features of selective interaction with a particular protein or proteins and be bioavailable and/or able to penetrate target cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

Typically, drug-like compounds or lead compounds are selected by their effect in the screening methods or assays compared to no compound or a control compound.

A further aspect of the invention provides a compound identified by the method of any of the tenth or eleventh or twelfth aspects of the invention.

Preferably, the compound is packaged and presented for use in medicine, or the compound is prepared into a pharmaceutical formulation comprising an effective amount of the compound and a pharmaceutically acceptable carrier, some of which are described in more detail above.

A still further aspect of the invention provides a method of treating CF the method comprising administering to a patient a compound which modulates nucleoside diphosphate kinase activity or a compound which modulates histidine phosphorylation of annexin or a compound which modulates the interaction between any of cystic fibrosis transmembrane conductance regulator protein (CFTR), nucleoside diphosphate kinase (NDPK) and annexin. Preferably, the compound modulates phosphorylation of histidine 246 or histidine 293 of annexin.

As is described in the Examples, we have found that annexin is phosphorylated on histidine 246 or histidine 293. Peptides which encompass these histidine residues are useful, for example in preparing reagents which are useful in raising antibodies.

A further aspect of the invention provides a peptide of relative molecular mass less than 6500 comprising at least five consecutive residues surrounding histidine 246 of annexin. Preferably, the peptide comprises at least 6 or 7 or 10 or 15 or 20 consecutive residues surrounding histidine 246 of annexin.

A still further aspect of the invention provides a peptide or relative molecular mass less than 6500 comprising at least five consecutive residues surrounding histidine 293 of annexin. Preferably, the peptide comprises at least 6 or 7 or 10 or 15 or 20 consecutive residues surrounding histidine 293 of annexin.

Preferably, the said histidine residue in either type of peptide is phosphorylated.

Methods of making peptides are disclosed above in relation to the CFTR peptides.

A further aspect of the invention provides a method of raising an antibody reactive with histidine phosphorylated annexin, the method comprising using a phosphorylated peptide as described above as an immnunogen. Typically, the phosphorylated peptide is combined with a carrier or an adjuvant or both.

Peptides in which one or more of the amino acid residues are chemically. modified, before or after the peptide is synthesised, may be used providing that the function of the peptide, namely the production of specific antibodies vivo, remains substantially unchanged. Such modifications include forming salts with acids or bases, especially physiologically acceptable organic or inorganic acids and bases, forming an ester or amide of a terminal carboxyl group, and attaching amino acid protecting groups such as N-t-butoxycarbonyl. Such modifications may protect the peptide from in vivo metabolism. The peptides may be present as single copies or as multiples, for example tandem repeats. Such tandem or multiple repeats may be sufficiently antigenic themselves to obviate the use of a carrier. It may be advantageous for the peptide to be formed as a loop, with the N-terminal and C-terminal ends joined together, or to add one or more Cys residues to an end to increase antigenicity and/or to allow disulphide bonds to be formed. If the peptide is covalently linked to a carrier, preferably a polypeptide, then the arrangement is preferably such that the peptide of the invention forms a loop.

According to current immunological theories, a carrier function should be present in any immunogenic formulation in-order to stimulate, or enhance stimulation of, the immune system. It is thought that the best carriers embody (or, together with the antigen, create) a T-cell epitope. The peptides may be associated, for example by cross-linking, with a separate carrier, such as serum albumins, myoglobins, bacterial toxoids and keyhole limpet haemocyanin. More recently developed carriers which induce T-cell help in the immune response include the hepatitis-B core antigen (also called the nucleocapsid protein), presumed T-cell epitopes such as Thr-Ala-Ser-Gly-Val-Ala-Glu-Thr-Thr-Asn-Cys, beta-galactosidase and the 163-171 peptide of interleukin-1. The latter compound may variously be regarded as a carrier or as an adjuvant or as both. Alternatively, several copies of the same or different peptides of the invention may be cross-linked to one, another; in this situation there is no separate carrier as such, but a carrier function may be provided by such cross-linking. Suitable cross-linking agents include those listed as such in the Sigma and Pierce catalogues, for example glutaraldehyde, carbodiimide and succinimidyl 4-(N-maleimidomethyl)cyclohexane-1-carboxylate, the latter agent exploiting the —SH group on the C-terminal cysteine residue (if present).

The peptide of the invention may be linked to other antigens to provide a dual effect.

The invention will now be described in more detail with reference to the following Figures and Examples wherein

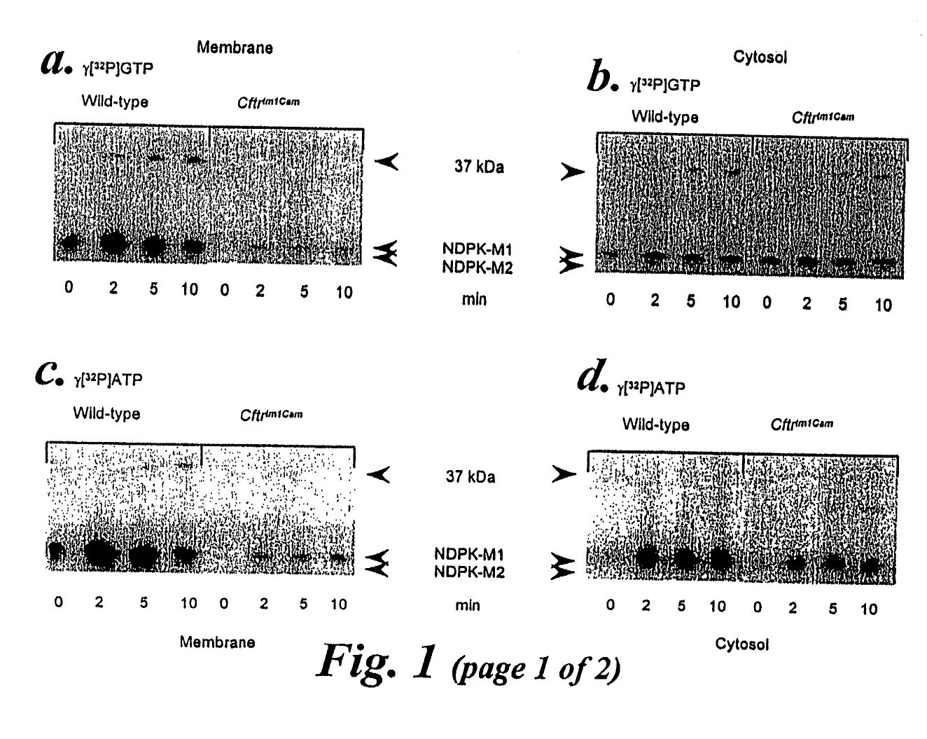

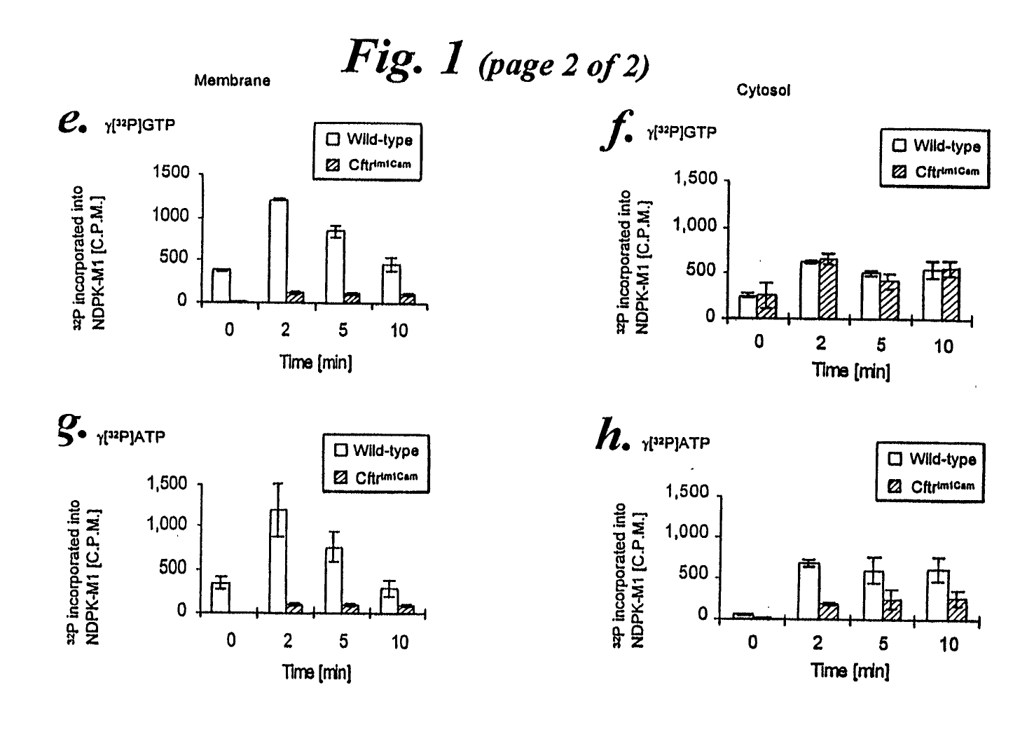

FIG. 1. Comparison of phosphorylation of NDPK between wild-type and Cftr^{tm1Cam} murine tracheal epithelia with different kinase substrates. Panels show electronic autoradiographs of time-course (0-10 min) of phosphorylation in membrane and cytosolic preparation using γ[³²P]GTP (a, b) or γ[³²P]ATP (c, d) at 37° C. Membranes (a, c): Phosphorylation of NDPK was attenuated in Cftr^{tm1Cam} compared to wild-type irrespective of nucleotide substrate. Quantitation (e, g) confirmed a significant reduction in phosphorylation (p<0.01; mean±range, n=3). Cytosol (b, d): Phosphorylation of NDPK in Cftr^{tm1Cam} defective phosphorylation of cytosolic NDPK was confined to ATP as kinase substrate (quantitation f, h, for Cftr^{tm1Cam} ATP p<0.01; mean±range, n=3).

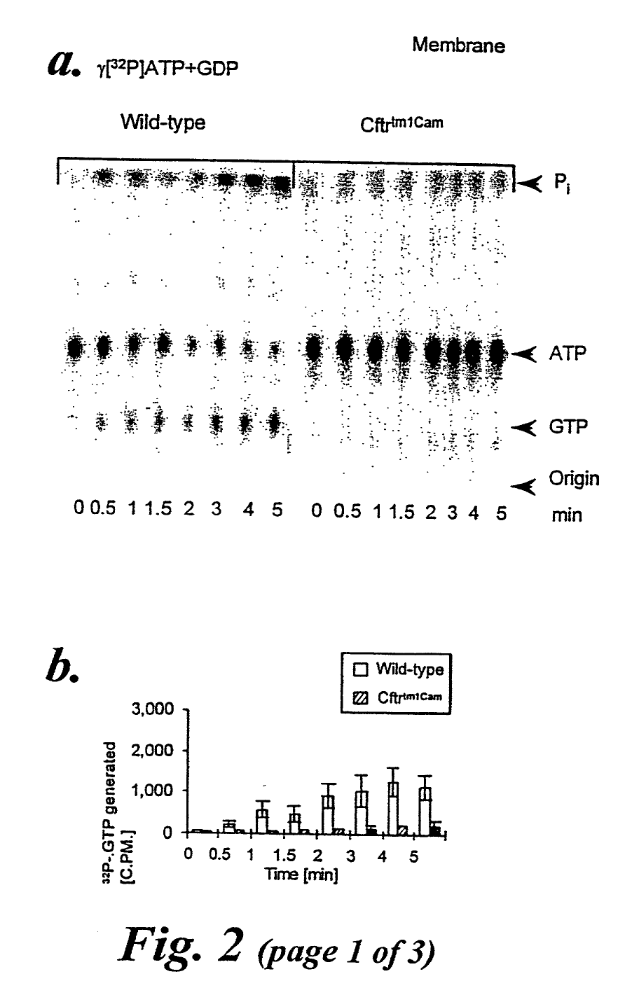

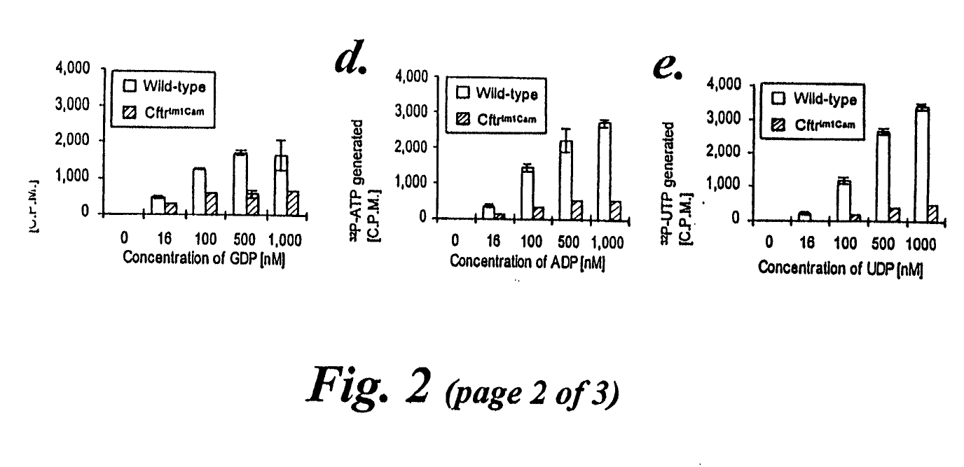

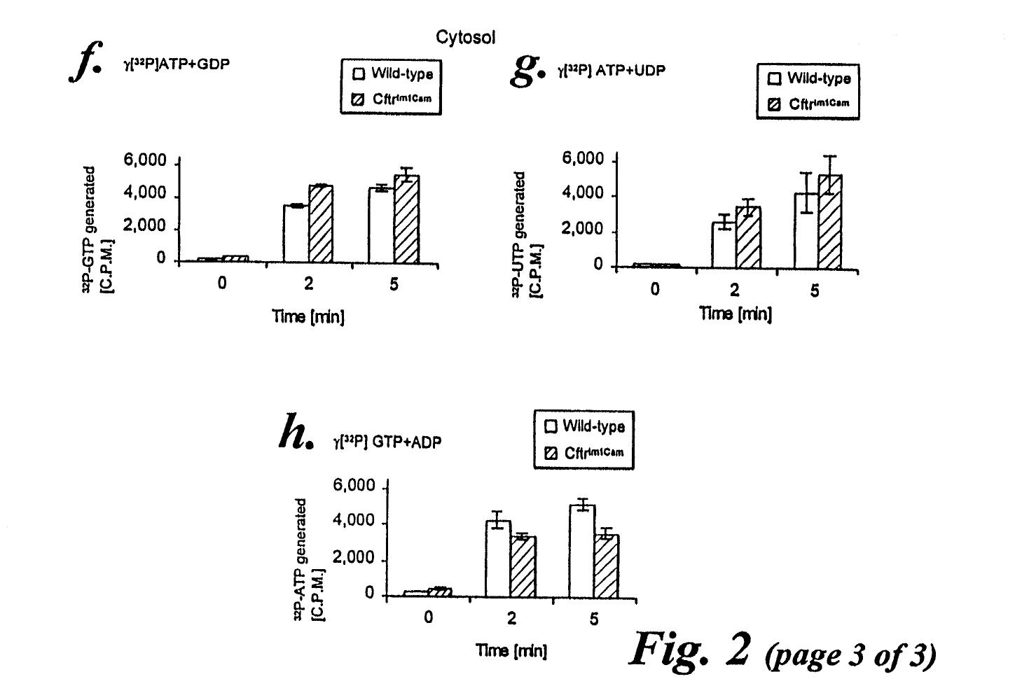

FIG. 2. Comparison of nucleotide synthesis in Cftr^{tm1Cam} and wild-type mice. Membranes: a-e, Electronic autoradiograph of TLC plate (a) compares time-course of synthesis of γ[³²P]GTP from γ[³²P]ATP+GDP (500 nM at 37° C. Enhanced GTP production occurred in wild-type but not Cftr^{tm1Cam} (n=3±range, for quantitation see b). Despite excess dinucleotide substrate and irrespective of the dinucleotide - trinucteotide combination panels c-e show that phosphotransferase activity was significantly reduced (p<0.01) in membranes from Cftr^{tm1Cam} compared to wild-type (attenuation of UTP>ATP >GTP). Cytosol: f-h, Comparison of ATP, GTP and UTP synthesis shows no significant differences between wild-type and Cftr^{tm1Cam} cytosol (n=3±range p>0.05).

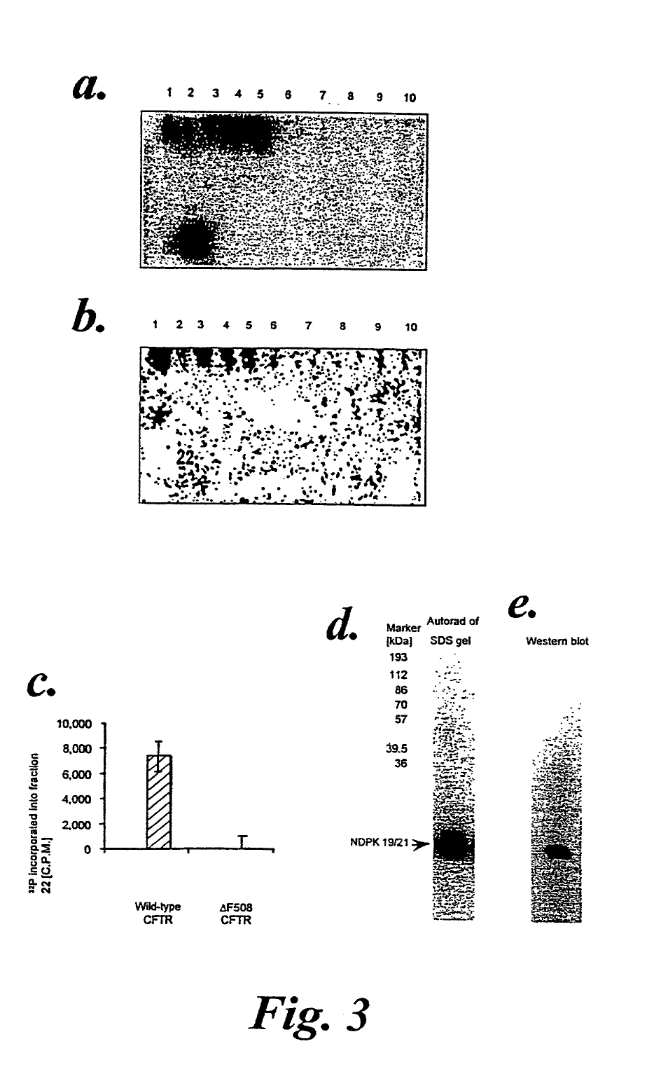

FIG. 3. Detection of NDPK protein in airway epithelia of Cftr^{tm1Cam}/wild-type mice and restoration of function by transfection. a, Antibodies specific to NDPK immunoprecipitated a phosphorylated 21 kDa protein from wild-type murine membrane preparation which had been pre-phosphorylated with γ[³²P]ATP. Addition of specific peptide blocked this precipitation. b, These antibodies detected a 21 kDa protein in membrane and cytosol from both Cftr^{tm1Cam} and wild-type mice. The membrane and cytosol from Cftr^{tm1Cam} mice showed a higher content of NDPK compared to wild-type (contrast the phosphorylation defect FIG. 1). c, d Transfection with plasmid±CFTR (pTRIAL10CFTR2 or pTRIAL10, with and without CFTR, respectively)²⁵ shows restoration of NDPK phosphorylation (c) and nucleotide production (d) only when CFTR was present. That plasmid transfection alone also increased NDPK phosphorylation slightly above normal control could be explained by deranged nucleotide metabolism during transfection since NDPK is central to cellular nucleotide exchange.

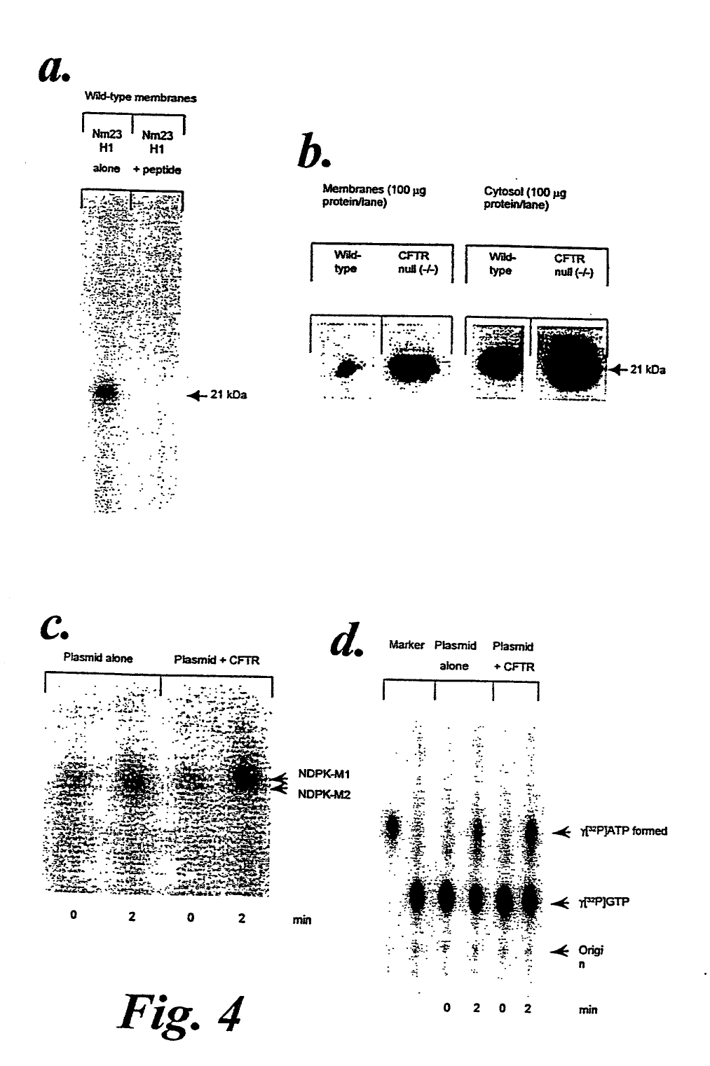

FIG. 4. Evidence for interaction between CFTR and NDPK. Electronic autoradiographs of slot blots showing eluted human nasal membrane proteins from either wild-type (a) or mutant ΔF508 (b) peptide-affinity columns. The blots were exposed to Δ[³²P]ATP and a phosphoprotein was observed in fraction 22 from the wild-type but not mutant peptide column (quantitation, c). d, Phosphorylation of eluate from ovine CFTR peptide column showed a 19/21 kDa phosphoprotein following electronic autoradiograph of SDS-PAGE. e, ECL-Western blot analysis of the eluate from ovine CFTR peptide column using antibodies specific to NDPK detected a 21 kDa protein.

FIG. 5. Purification and histidine phosphorylation of annexin I (Anx I). a, SDS-PAGE of an aliquot of the original ovine membrane fraction lane 1), anion exchange flow through fraction lane 2) and a concentrated fraction (lane 3) showing the concentration of a major band of 37 kDa with a minor 70 kDa band. Electronic autoradiographs b, c: b, Antibodies specific to Anx I (but not control Gβ subunit of G proteins) immunoprecipitated a phosphorylated 37 kDa protein from ovine membrane preparations which had been pre-phosphorylated with γ[³²P]ATP or GTP. c, immunoprecipitation of Anx I from wild type murine membranes phosphorylated with γ[³² P]ATP+GDP. d, protein sequence of bovine Anx I (GENBANK P46193) with peptide sequences (boxed) corresponding to peptides from tryptic digests of the 37kDa band (a, lane 03). The 70 kDa band also showed sequence identity with Anx I (*). e, electronic autoradiographs of SDS PAGE from ovine membrane proteins phosphorylated with γ[³²P]ATP for 5 min and then treated with 0.1M HCl (lane 1), 0.1M NaOH (lane 2), 0.1M acetic acid/hydroxylamine (lane 3) or sham-treated time control (lane 4) at 30° C. for 10 min. The phosphorylation of Anx I is acid labile. f, HPLC phosphoamino acid analysis of phosphorylated Anx I p37 - e, lane 4) shows co-elution of radioactivity (bold line) with phosphohistidine standard (peak c).

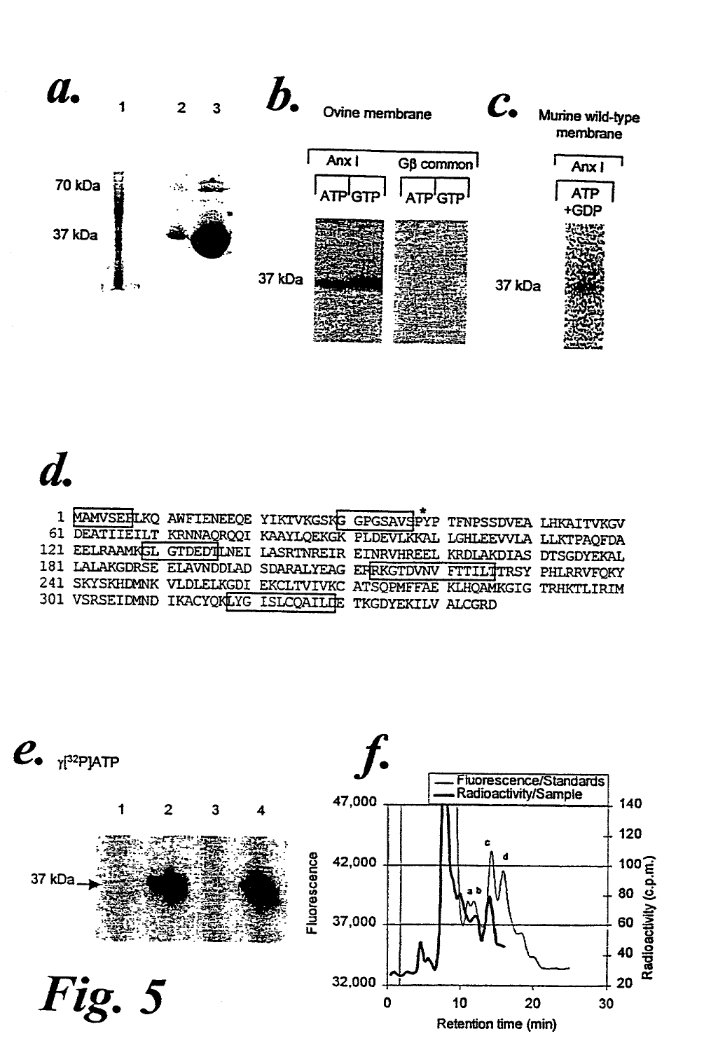

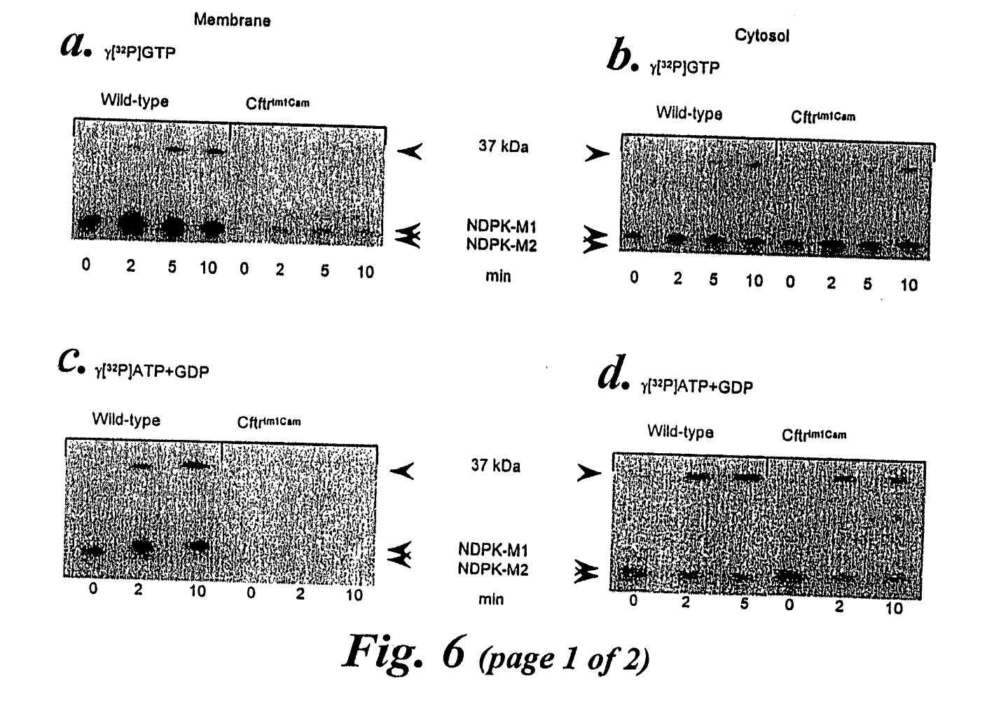

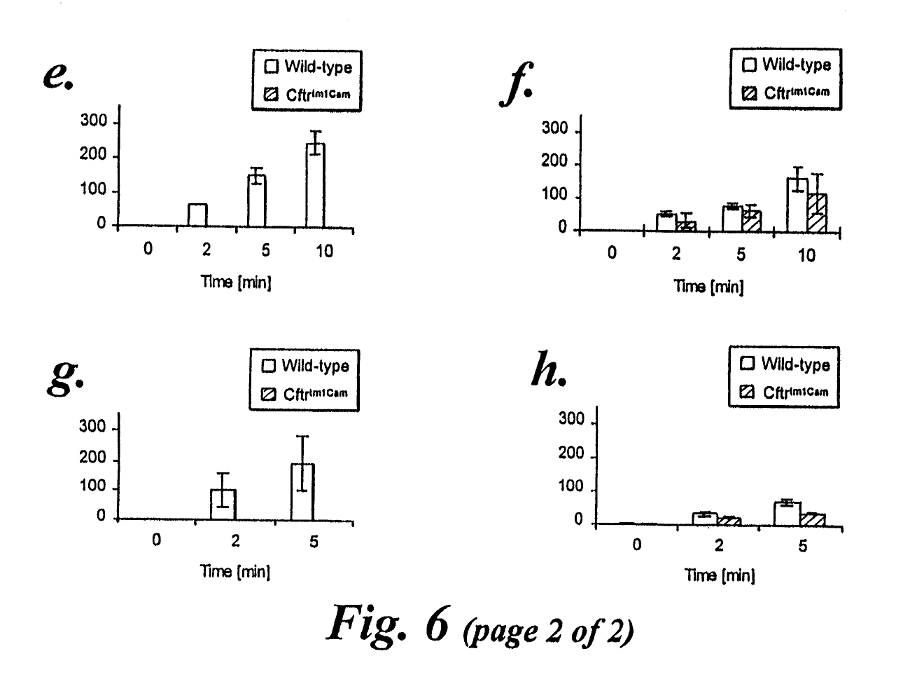

FIG. 6. Comparison of phosphorylation of annexin I (Anx I) between wild-type and Cftr^{tm1Cam} murine tracheal epithelia. Panels show electronic autoradiographs of time-course (0-10 min) of phosphorylation in membrane and cytosolic preparations using γ[³²P]GTP (a, b) or γ[⁼P]ATP+GDP (c, d) at 37° C. Membranes (a, c): Phosphorylation of Anx I was attenuated in Cftr^{tm1Cam} compared to wild-type. Quantitation (e, g) confirmed an absence of phosphorylation of Anx I (p<0.01; mean±range, n=3). Cytosol (b): With γ[³²P]GTP as kinase substrate, phosphorylation of Anx I in Cftr^{tm1Cam} was not significantly different from wild type (quantitation f, p<0.01; mean±range, n=3). In contrast to the membrane, the addition of GDP to γ[³²P]ATP resulted in a partial restoration of Anx I phosphorylation (h, compare g).

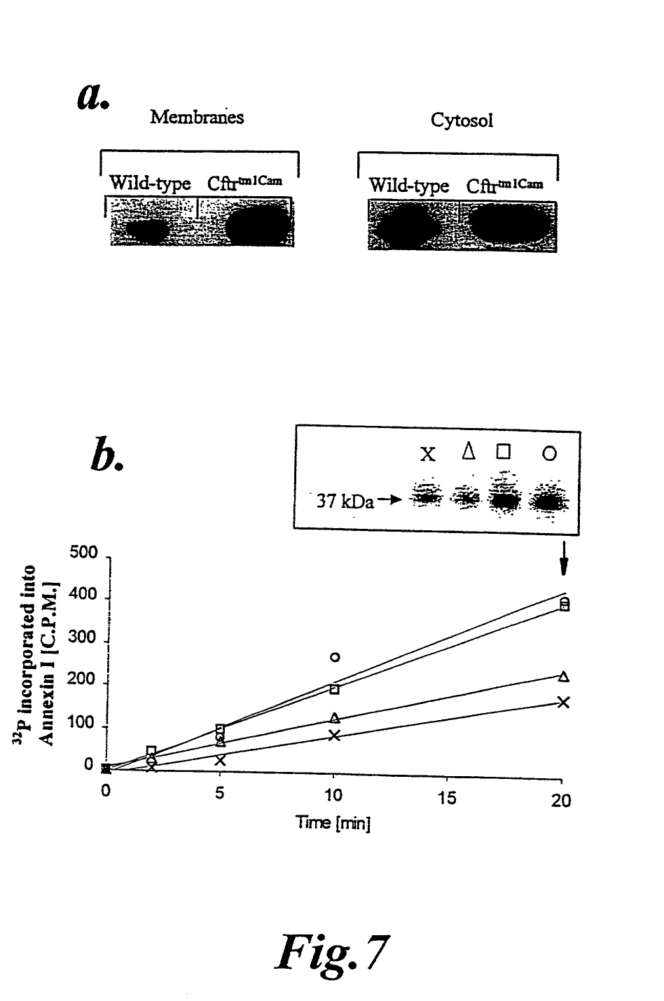

FIG. 7. Detection of annexin I protein in airway epithelia of Cftr^{tm1Cam}/wild-type mice and restoration of phosphorylation in Cftr^{tm1Cam} mice by transfection with a CFTR expression plasmid. a, Antibodies to Anx I detected a 37 kDa protein on Western blots using membrane and cytosol from both Cftr^{tm1Cam} and wild-type mice (representative blot, n=3). Compared to wild-type, both membrane and cytosol from Cftr^{tm1Cam} mice showed a higher content of Anx I protein. b Time course (0-20min, mean of 3 replicates) of Anx I phosphorylation following transfection (pTRIAL10CFTR2(□□) or pTRIAL10(Δ)±CFTR, respectively)²⁸ shows restoration of Anx I phosphorylation to wild type (o) only when CFTR was present. Cftr^{tm1Cam} control (X) is shown for comparison and the inset shows the electronic autoradiograph for each limb at 20 min.

FIGS. 8 to 13. These show various autoradiographs of ³²P-labelled proteins from membranes, separated by SDS-PAGE, of wild-type and null CFTR mice in which the phosphorylation state of various proteins is different in wild-type compared to null CFTR mice. The protein marked p37 is annexin. The phosphorylation of p116 is decreased in null CFTR mice compared to wild-type mice. The phosphorylation of p11 is increased in Cftr^{tm1Cam} mice compared to wild-type.

FIG. 14. Localisation of phosphohistidine to the core of annexin I. A partially purified annexin I preparation pre-phosphorylated with γ[³²P] GTP was incubated in the presence of Ca²⁺ (4 mM) for 30 min at 37° C. This generated a phosphorylated band of 21 kDa suggesting cleavage of annexin I by an endogenous: protease. Coomassie staining showed the presence of two protein bands (circa 18 and 21 kDa, not shown) only one of which contained the phosphate label. Both bands were sequenced and the upper of the two, which corresponded to the phosphorylated band, contained an amino acid sequence corresponding to the C terminal half of the annexin I core (panel b). (The sequence of the lower corresponded to the N-terminus of the protein, not shown) An antibody directed against a C terminal annexin I peptide was used to confirm the identity of the phosphorylated band (panel a). Panel C shows candidate histidine residues from this region of annexin I (stars indicate histidine residues conserved across, species). GENBANK accession numbers for the sequences shown are: rabbit—P51662; human—P04083; bovine—P46193; mouse—P10107.

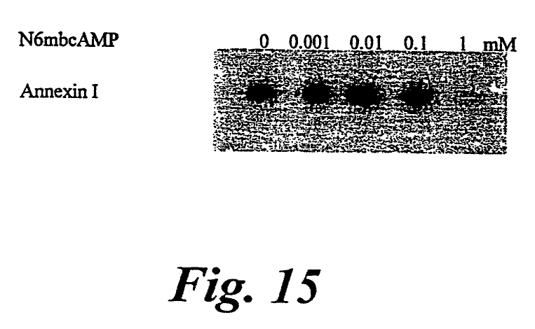

FIG. 15. Inhibition of annexin I phosphorylation by N-mbcAMP. A membrane preparation pre-incubated at 4° C. for 30 min with N⁶-monobutyryladenosine 3′:5′ cyclic monophosphate (N⁶-mbcAMP) (0-1mM) was phosphorylated with γ[³²P] ATP at 37° C. for 5 min. Maximal inhibition of histidine phosphorylation of annexin I occurred at 1 mM.

### Example 1

Nucleoside diphosphate kinase function is dependent on the cystic fibrosis transmembrane conductance regulator

Cystic Fibrosis (CF) results from mutations in the cystic fibrosis transmembrane-conductance regulator protein (CFTR) which controls epithelial chloride transport. However, the multiple regulatory disorders in CF cells cannot be reconciled with defective chloride transport alone. This Example shows that a multi-functional kinase (nucleoside diphosphate kinase, NDPK), is defective in transgenic mice not expressing CFTR, but functionally restored following CFTR gene therapy in vivo. NDPK binds to wild-type (but not disease-associated) peptides corresponding to the CFTR region carrying the common disease mutation. These findings suggest a mechanism whereby mutant CFTR protein produces CF disease.

Cystic Fibrosis (CF) is caused by mutations in the cystic fibrosis transmembrane-conductance regulator (CFTR) which forms a cell-surface chloride channel in epithelia (1). However, the multiple disorders in CF cell physiology are difficult to reconcile with defective chloride transport alone. These include impaired exocrine gland function (2) and essential fatty acid deficiency (3) accompanied by excess sodium transport (4), phospholipase A₂ activity (5) and pulmonary neutrophil accumulation without infection (6). Amongst these pleiotropic CF defects, disordered GTP-dependent secretion and disrupted chloride transport are unexplained recurring themes. We have described a GTP-utilizing, chloride-sensitive phosphorylation cascade whose function is currently unknown (⁷⁻⁹). The cascade is present in apical membranes from airway epithelia in humans (^{8,9}), sheep (⁷) and mice (¹⁰. Amongst the proteins within this cascade, the phosphorylation of a 19/21 kDa doublet is sensitive to [Cl⁻] with maximal phosphorylation occurring at physiologically relevant intracellular chloride concentrations (^{7,9}). This phosphorylation profile is additionally cation—and nucleotide-dependent with sodium (>10 mM) selectively inhibiting the phosphorylation of these proteins when ATP (but not GTP) is the phosphate donor (⁸). We identified the doublet proteins as membrane-bound isoforms of an enzyme which regulates many processes including tumour metastasis and embryogenesis—nucleoside diphosphate kinase (NDPK) (-) (de Ia Rosa et al (1995) BioEssays 17, 53-62). NDPK forms a hexameric heteromultimer in vivo containing two closely related isoforms (M1/M2 in mice, Nm23H1/H2 in humans) (¹¹). NDPK has two discrete modes of action whose inter-relationship is not understood. In addition to its role as a protein kinase (substrates include enzymes involved in fatty acid synthesis and energy utilisation) (^{12, 13}), NDPK confers nucleotide selectivity to multiple cellular pathways (¹⁴) by converting di- to tri-nucleotides e.g. ATP+UDP→ADP+UTP via a high energy phosphohistidine intermediate.

Since [Na⁺] and [Cl⁻] are interactive regulators of NDPK phosphorylation, we hypothesised that NDPK dysfunction could contribute to the pleiotropic changes observed in CF. We confirmed the presence of phosphorylated NDPK in murine plasma membranes (¹⁵) by immunoprecipitation (see below) and then characterized NDPK phosphorylation in wild-type and mutant Cftr^{tm1Cam} mice (which express no CFTR protein). In wild-type mice, we observed two differences in NDPK compared to previously characterised ovine and human isoforms. Firstly, the M1 (FIG. 1, 21kDa isoform) form of NDPK was more phosphorylated than its M2 homologue and secondly, the two isoforms showed very little spatial resolution on SDS-PAGE. Phosphorylation of both isoforms is defective in plasma membranes from airway epithelia of Cftr^{tm1Cam} mice. Maximal phosphorylation of NDPK was attenuated 10 fold in Cftr^{tm1Cam} airway membranes relative to wild-type (FIG. 1a, c) (for quantitation of NDPK M1, see FIG. 1e, g; M2 quantitation data similar to M1, not shown). Since phosphorylated NDPK is an essential intermediate in the transfer of enzyme-bound phosphate to the incoming di-nucleotide, loss of phosphorylated NDPK predicted that Cftr^{tm1Cam} membranes would fail to synthesize tri-phosphate nucleotides. Nucleotide analysis (¹⁰) using an aliquot (1 μl) of the phosphorylation reactions (see FIG. 1), confirmed that despite the presence of excess di-nucleotide (ADP, GDP or UDP) acceptor, Cftr^{tm1Cam} membranes are unable to augment the synthesis of their corresponding triphosphate nucleotides (FIG. 2, a-e). This defect was not due to loss of NDPK protein because Western blots showed that NDPK was not only present in both membrane and cytosolic preparations from Cftr^{tm1Cam} mice but was actually increased in quantity (semi-quantitative) compared to wild-type controls (FIG. 3b; see also FIG. 3a for immunoprecipitation of phosphorylated NDPK from wild type). The above results suggest that NDPK dysfunction may account for some of the defects observed in CF.

Because NDPK trafficks between cytosol and membrane, we studied its activity in the cytosol of mouse epithelia. Cytosolic nucleotide production was unaffected by loss of CFTR because ATP, UTP and GTP generation were indistinguishable from wild-type (FIG. 3f-h). However, we observed a discrete reduction in phosphorylation (three fold) of cytosolic NDPK M1 from Cftr^{tm1Cam} mice with ATP (but not GTP) (FIG. 1 compare h, f). Since sodium (>10 mM) promotes dephosphorylation of NDPK when ATP (but not GTP) is present (⁸), the observed loss of ATP-dependent phosphorylation of both membrane-bound and cytosolic NDPK suggests that the feedback loop involving a ‘sodium sensor’ is defective in CF. Our recent data (⁸) suggest that the accompanying anion modulates the degree of sodium-dependent inhibition (chloride>gluconate), thus linking chloride to the defective sodium loop. An excess trans-apical absorption of sodium occurs in CF airway epithelium (⁴) and CF patients have a low body mass index and fatty acid deficiency which may contribute to this disordered sodium transport (⁵) by unknown mechanisms. We note that ATP-citrate lyase, the enzyme controlling the rate limiting step of fatty acid synthesis, is directly phosphorylated by NDPK (¹²). Although the functional consequences of this interaction are unknown, it is possible that NDPK dysfunction will disrupt his process. This may not be the sole defect because NDPK both co-purifies with, and supplies ATP to the succinyl-CoA synthetase complex (¹³), a mitochondrial en e controlling the supply of fatty acid precursors to the cytosol.

The above data show that the defects involving nucleotide synthesis and phosphorylation of NDPK are linked to the absence of CFTR in Cftr^{tm1Cam} tracheal epithelium. We predicted that these defects would not be restricted to the murine CF model and conducted experiments using membrane and cytosol preparations from a homozygous ΔF508 CFTR airway cell line (CFBE). Our results show that when compared to equivalent wild-type 16HBE cells, CFBE cell membranes have a 50% reduction in both nucleotide synthesis (c.p.m. GTP generated - 16HBE, 3446±115 and CFBE, 1640±44; n=3±range) and NDPK phosphorylation (c.p.m. phosphate incorporated into NDPK-H1- 16HBE, 232±84 and CFBE, 102±59; n=3±range). This result shows a cross-species dysfunction of NDPK when CFTR is either mutated or absent.

The β-pleated sheet in the region of the most common mutation at position 508 has been found to be unstable when F508 is deleted (¹⁶). Recent structural predictions suggest that this unstable region lies outside the nucleotide binding fold (¹⁷). We speculated that this part of CFTR would associate with protein(s) from the chloride-sensitive phosphorylation cascade. We constructed peptides corresponding to the CFTR region encompassing position 508 of human CFTR. Membrane proteins from wild-type human nasal epithelium were incubated with either of two peptides corresponding to the wild-type or the ΔF508 mutant form of CFTR (KENIIFGVSYDEYR or KENIIGVSYDEYR, respectively) linked to an affinity matrix (¹⁸). The wild-type peptide column bound protein(s) that could be phosphorylated with β[³²P]ATP when blotted onto nitrocellulose (FIG. 4a), but no labeling was seen with α [³²ATP (data not shown). No phosphorylation was observed in eluates from the ΔF508 mutant peptide column (FIG. 4b). In order to generate sufficient material to characterise the bound protein(s), this experiment was repeated using the equivalent ovine wild-type CFTR peptide (KDNIIFGVSYDEYR) and membrane proteins from ovine tracheal epithelia (¹⁸) Proteins eluted from this peptide column were phosphorylated with γ[³²P]ATP and separated by SDS-PAGE. Electronic autoradiography showed that a 19/21 kDa doublet was the sole phosphorylated species in the eluate FIG. 4(d). Western blot analysis detected a 21 kDa protein, indicating the presence of NDPK in this fraction. The above data suggests that NDPK interacts with CFTR at the site of the common mutation F508 provided phenylalanine is present. Because ΔF508 CFTR protein, when reconstituted into a lipid bilayer (¹⁹) shows normal chloride channel activity, it has been proposed that measures aimed at increasing the release of mutant ΔF⁵⁰⁸ protein from the ER to the apical membrane may alleviate the symptoms of CF. However, the above data coupled to the defective phosphorylation of NDPK in the CFBE cells suggests that monotherapy may not be sufficient for full correction of the cellular pathology in CF. In contrast, the failure of membrane-delimited UTP production in CF does provide an additional rationale for the administration of UTP in this disease.

Current CF gene therapy strategies are directed towards the introduction of a normal copy of the CM gene into epithelia to restore defective cAMP-dependent chloride secretion. Previous studies have shown that delivery of CFTR cDNA into airway epithelia of Cftr^{tm1Cam} mice results in transient restoration of cAMP-dependent Cl⁻ transport at 2 days post lipofection (¹⁵). Phosphorylation assays using membranes prepared from murine trachea transfected with CTFR showed that both NDPK phosphorylation and nucleotide synthesis were restored in Cftr^{tm1Cam} mice airway (FIG. 3c,d) two days after transfection. The restoration of NDPK phosphorylation under conditions that have been shown previously to transiently restore Cl⁻ transport in Cftr^{tm1Cam} mice suggests alternative, techniques for assessment of the clinical efficacy of both CF gene therapy and CF pharmacotherapy.

In conclusion, our data shows that in airway epithelia, CFTR interacts with NDPK and that NDPK function in vivo is dependent on the presence of CFTR. Specifically, wild-type CFTR promotes phosphorylation of NDPK. We note that CFTR, a cAMP-activated chloride channel which is a regulator of diverse cellular processes, binds a cAMP-regulated NDPK (²⁰). NDPK manifests sodium, potassium and chloride-sensing capability and is known to regulate multiple cell signalling pathways (^{14,21-26}). Since NDPK also regulates neutrophil-mediated inflammation (^{27,28}) and CF lungs accumulate neutrophils in the absence of infection (⁶), NDPK dysfunction unites features of CF which have hitherto been inexplicable. The coalescence of integral and membrane associated proteins may include other unidentified proteins (e.g. the chloride-sensitive p37, see FIG. 1) within this phospho-relay whose functions are collectively disordered in the absence CFTR.

References for Example 1, including methods

1. Kerem, B. S. et al (1989) Science 245, 1073.

2. McPherson, M. A. & Dormer, R. L. (1987) Bioscience Reports 7, 167.

3. Christophe, A. & Robberecht, E. (1996). Prostaglandins, leukotrienes and Essential Fatty Acid 55, 129.

4. Boucher, R. C. et al (1988) J Physiol. (London) 405, 77.

5. Strandvik. B. Svensson, E. & Seyberth, H. W. (1996) Prostaglandins, leukotrienes and Essential Fatty Acids 55, 419.

6. Khan, T. Z. et al (1995) Am. J. Resp. Crit. Care Med. 151, 1075.

7. Muimo, R. Banner, S. Marshall, L. J. & Mehta, A. (1998) Am. J. Respir. Cell. Mol. Biol. 18, 270.

8. Marshall, L. J. Muimo, R. Riemen, C. E. & Mehta, A. (1999) Am. J. Phys. Cell. 276 (Cell Physiol 45) C109-119.

9. Treharne, K. J. Marshall, L. J. & Mehta, A. (1994) Am. J. Physiol. (Lung Cell. Mol. Physiol.) 267, L592.

10. Riemen, C. E. Muimo, R. Ratcliff R. A. Colledge, W. H. Evans, M. J. & Mehta, A. (1998) J. Physiol. 509 81P.

11. Parks Jr, R. E. & Agarwal, R. P. Nucleoside diphosphokinases. In the enzymes. Boyer, P. D. 307-331. (Academic Press, New York, 1973).

12. Wagner, P. & Vu, N. D. (1995) J. Biol. Chem. 270, 21758.

13. Kadrmus, E. F. Ray, P. D. & Lambeth, D. O. (1991) Biochem Biophys. Acta 1074, 339.

14. Engel, M. et al (1998) J. Biol. Chem. 273, 20058.

15. Wild-type and mutant Cftr^{tm1Cam} mice (6-10 and 4-12 weeks old, respectively) were killed by exposure to CO₂ in accordance with institutional guidelines as described [Ratcliff, R. et al. Nature Genet. 4, 35 (1993)]. Pooled (at least 10 tracheae/prep; 4 tracheae for each part of the transfection experiments [Goddard, C. A. et al. Gene Therapy 4, 1231 (1997)]) tracheal scrapings (4° C.) were manually homogenised for 1.5 min, spun at 600×g (5 min), supernatant respun at 100,000×g (2 h) and the pellet resuspended and spun thrice at 16,000×g (30 min) to prepare a lactate dehydrogenase (LDH)-free pellet (10,7).

16. Thomas, P. J. et al (1992) J. Biol. Chem. 267, 5727.

17. Armstrong, S. et al (1998) Pediatr. Pulmanol. Suppl. 17, 91.

18. Human nasal epithetium: Apical membrane proteins from human nasal epithelium were solubilised at 4° C. (10 mM KOH, 0.1% Tween), dialysed against PBS-Tween containing protease inhibitors (3 h) and spun at 150 000 g for 1 h. The supernatant was then re-circulated over affinity supports containing either synthetic peptide KENIIFGVSYDEYR or KENIIGVSYDEYR, corresponding to the F508 region of wild-type or γF508 mutant CFTR, respectively. The columns were washed with PBS-Tween (0.1%) and eluted with 1M KI. Fractions (40×1 ml) were slot blotted onto nitrocellulose under vacuum. The blots were washed in buffer A (10 mM MOPS pH 7.9, 1 mM MgCl₂, 0.05% Triton X-100, 1 mM ATP) for 10 min and subsequently labelled with 5 nM γ[³²P]ATP (specific activity 222 TBq/mmol) in buffer A (1 h at room temperature). The blots were finally washed with PBS-Tween (0.1%) for 30 min (6 buffer changes) and visualised using electronic autoradiography. Sheep tracheal epithelia. The pelleted membranes from sheep tracheal epithelia were solubilised with 0.1% glucopyranoside in 50 mM HEPES pH 7.4 on ice for 1 h and centrifuged at 100 000×g for 30 min. The supernatant was applied to a peptide affinity column (KDNIIFGVSYDEYRC, corresponding to the F508 region of ovine wild-type CFTR, coupled to epoxy activated POROS 20 media using the additional terminal cysteine residue). The column was washed with load buffer containing 1M NaCl, followed by load buffer only (100 column volumes or stable OD₂₈₀˜0.015). Bound proteins were eluted with 50 mM glycine-NaOH pH 11 containing 0.1% glucopyranoside and immediately neutralised with 1M Tris-HCl pH 8.0.

19. Li, C. et al (1993) Nature Genet. 3, 311.

20. Anciaux, K. et al (1997) FEBS Lett. 400; 75.

21. Kikkawa, S. et al (1990) J. Biol. Chem. 265, 21536.

22. Kimura, N. & Shimada, N. Biochem. Biophys. Res. Com. (1990) 168, 99.

23. Komwatana, P. et al (1996) Proc. Natl. Acad. Sci. USA 93, 8107.

24. Wieland, T. et al (1991) Eur. J. Pharmacol. 208, 17.

25. Marciniak, S. J. & Edwardson J. (1996) M. Biochem. J. 316, 99.

26. Xu, L. Murphy, J. & Otero, A. (1996) J. Biol. Chem. 271, 21120.

27. Blevins Jr, G. T. Van Der Westerlo, E. M. A. & Williams, J. A. (1994) Am. J. Physiol. 30, G866.

28. Guignard, F. & Markert, M. (1996) Biochem. J. 316, 233.

### Example 2

Histidine phosphorylated Annexin I links inflammation and secretion in cystic fibrosis

Cystic fibrosis (CF) results from mutations in the cystic fibrosis transmembrane conductance regulator (CFTR) which forms a cAMP-regulated epithelial chloride channel¹. However, CF cell physiology is so complex that CFTR must have functions beyond the control of chloride conductance. CF also manifests dysregulated cell secretion^{2,3} with excess inflammation⁴ and pulmonary neutrophil accumulation in the absence of infection⁵. The annexin (Anx) gene family⁶ shares sequence homology with CFTR⁷ and, by unknown mechanisms, one family member (Anx I) regulates the above processess⁸⁻¹². Here, we show that a novel histidine phosphorylation of Anx I, is dependent on the presence of CFTR in vivo. Compared to wild-type mice, Anx I phosphorylation is attenuated in CFTR-null (Cftr^{tm1Cam)}¹³ tracheal epithelium but restored following in vivo transfection with a cftr expression plasmid. The CFTR-dependence of Anx I histidine phosphorylation suggests that annexin dysfunction plays a pivotal role in the pathogenesis of CF lung disease. Our work defines CF as the first mammalian disease in which histidine phosphorylation is disordered and provides a mechanism to explain annexin function.

Anion substitution is a universal stratagem for study of vectorial chloride transport across epithelia. The replacement anion may, however, itself alter membrane protein structure¹⁴ and hence affect the interpretation of the results. We have described an ion-sensitive phosphorylation cascade on apical membrane of airway epithelium^{14,15,16} which contains a 37 kDa protein (p37) whose phosphorylation state alters with both anion species and concentration ^{14,15}. Because p37 was the only major phosphorylated protein when membrane proteins from ovine airway epithelia are incubated with ATP and GDP¹⁵, we used metal chelate (iminodiacetic acid-Fe³⁺) affinity chromatography¹⁷ to purity phosphorylated p37 to homogeneity (FIG. 5a). Sequence analysis of “in-gel” peptide digests from the purified product showed 100% homology to bovine Anx I (FIG. 5d). In order to prove that p37 and Anx I were identical, pre-phosphorylated ovine p37 was immunoprecipitated with antibodies raised against Anx I (FIG. 5b). Since Anx I regulates endocytosis, we propose that the [chloride] sensitivity of Anx I phosphorylation may explain why this anion is essential for endosome fusion in airway epithelium³.

Previously, annexins have been reported, to be phosphorylated on serine and tyrosine residues within the N-terminus by PKC and EGF receptor kinase, respectively¹⁸ but acid hydrolysis (6 N HCl at 105° C., 2 h) of ovine [³²P]Anx I. (labelled for 5 min) removed>95% of the radioactivity from p37 (Anx I) (data not shown). Further analysis confirmed that this phospho-linkage was not on serine, threonine or tyrosine residues because it was acid labile (FIG. 5e, lanes 1 and 3), base stable (FIG. 5e, lane 2) and inhabitable following pre-incubation with diethylpyrocarbamate (modifies histidine residues, data not shown). This suggested a phospho-amidate linkage and HPLC-phosphoamino acid analysis of an alkali hydrolysate¹⁹ from ³²P]Anx I showed the presence of phosphohistidine (FIG. 5f). To our knowledge, this is the first report in annexins. Notably, all the histidine residues of Anx I lie within the calcium-dependent lipid-binding core (conserved structure between annexins) which indicates that this phosphorylation, the first out with the N-terminus of Anx I, may occur in other members of the annexin family. Histidine phosphorylation may regulate the proposed functions of the core—cation channel permeation²⁰ and/or membrane fusion^{6,8}. Consistent with this notion, the crystal structure of Anx I²¹ shows that histidines (with one exception) are asymmetrically distributed over the race of Anx I. These histidines are located on the calcium-binding face of the core which interacts with acidic lipid head groups phosphatidyl-serine/choline)⁶ on the plasma membrane.

Cyclic AMP controls both the membrane aggregating and the cation channel properties of Anx I via a proposed cAMP binding site²⁰. Because cAMP-dependent fluid secretion²² and vesicle trafficking^{2,3,22} are both disordered in CF airway and annexins share significant homology with the region of CFTR (amino acids 500-529) bearing the common disease mutation⁷, we speculated that annexins may be involved in CF. This hypothesis was consistent with the proposed anti-inflammatory role of Anx I via suppression of phospholipase A₂ activity^{10,24} and neutrophil migration^{9,10} particularly as both processes are upregulated in CF^{25,5}. We observed an absence of a 37 kDa phosphoprotein in plasma membranes from the airway of a CF mouse model, Cftr^{tm1cam}, which expresses no CFTR (FIG. 6a, quantitation FIG. 6e). We confirmed the identity of murine p37 as Anx I by immunoprecipitating pre-phosphorylated [³²P]p37 from wild type membranes using antibodies against Anx I (FIG. 5c). We have previously found that p37 (Anx I) phosphorylation is enhanced by GDP (but not GDPβS)¹⁵. This enhancement was also observed for wild type membranes in this study (FIG. 6c, quantitation in g) but the addition of ATP+GDP failed to either restore or enhance histidine phosphorylation of Anx I in Cftr^{tm1Cam} (FIG. 6c, g) suggesting a defect in the function(s) served by the protein. Because Anx I is a membrane-associated protein which shuttles between membrane and cytosol²⁶, we studied Anx I phosphorylation in cytosol from wild-type and Cftr^{tm1Cam}. In the absence of GDP and in contrast to the membrane, we found no differences in Anx I phosphorylation between wild-type and Cftr^{tm1Cam} cytosol (FIG. 6b; quantitation f). We confirmed the identity of cytosolic p37 as Anx I by immunoprecipitating pre-phosphorylated [³²P]p37 as described above (data not shown). In contrast to the membrane, addition of GDP to the Cftr^{tm1Cam} cytosol resulted in a partial restoration of Anx I phosphorylation (FIG. 6h, compare with 6g). The loss of Anx I phosphorylation in Cftr^{tm1Cam} membranes cannot be explained by absence of Anx I protein because Western blot analysis showed that Anx I was not only present but increased in both membrane and cytosol from Cftr^{tm1Cam} mice relative to wild-type (FIG. 7a). It is possible that the excess protein results from a compensatory increase in synthesis to overcome a disruption of function. Excess Anx I protein is consistent with the elevated plasma concentrations of the calcium-dependent annexin binding proteins (S100A8 and A9 or CF antigen) in both CF carriers and patients²⁷. S100 proteins form obligate dimers⁶ which bring together the N termini of different Anx I monomers to promote membrane-membrane fusion. Thus the CF paradox is an excess of both proteins coupled to dysregulation of cAMP-activated secretion, possibly induced by a disordered fusogenic function of the Anx I-(S100)₂-Anx I heterotetramer.

To confirm involvement of CFTR in the histidine phosphorylation of Anx I, airways of Cftr^{tm1Cam} mice were transfected, in vivo, with complementary CFTR or plasmid control²⁸. CFTR (but not plasmid) transfection restored annexin phosphorylation to wild type (FIG. 6b,c) suggesting that, in vivo, CFTR regulates histidine phosphorylation of Anx I. The plurality of processes regulated by annexins is unexplained⁶. That a channel protein with equally unexplained regulatory functions, interacts with Anx I via a process (histidine phosphorylation) which is important for cell signalling suggests an essential fiction, for Anx I in CF. In mammalian cells, the role of histidine phosphorylation is not understood but our results suggest that this phosphorylation is central to the normal function of CFTR and Anx I. We propose that Anx I histidine phosphorylation, which is [chloride]-dependent, links CFTR and disrupted, chloride-dependent endosome fusion in CF³.

Localisation of phosphohistidine on annexin I.

To further characterise annexin I phosphorylation, a 1,2-bis(2-aminophenoxy)ethane-N,N,N′,N′-tetraacetic acid (BAPTA) extract partially purified on chelating Sepharose Fe³⁺ column was phosphorylated with γ[³²P]GTP for 5 min, 37° C. and then incubated at 30° C. for 30 mm in the presence of calcium (4 mM). An additional 21 kDa phosphorylated band was observed. This result suggested cleavage of annexin I within the core domain due to Ca²⁺ activated proteases present in the sample (FIG. 14a). Sequence analysis of ‘in gel’ peptide digests from the 21 kDa band showed that this fragment corresponds to the carboxy terminal region of annexin I most likely covering annexin repeats 3 and 4 (FIG. 14b). The identity of the 21 kDa fragment with the C-terminal half of the annexin core was further confirmed using an affinity purified polyclonal antibody (Santa Cruz, C-19) raised to a peptide fragment from the C-terminal region of annexin I. This antibody recognises the phosphorylated 21 kDa fragment (FIG. 14a) and thus verifies that it is part of the C-terminal core domain of annexin I. Multiple sequence alignments with sequences from four species showed that only two of the three (or four depending on species) histidine residues (H246, H293) within this C-terminal domain are conserved (FIG. 14c).

Inhibition of phosphorylation by N⁶-mcbAMP.

Whilst characterising a histidine kinase, nucleoside diphosphate kinase (NDPK), we observed inhibiton of p³⁷ phosphorylation by cyclic AMP. Interestingly, cyclic AMP has been shown to bind to annexin I and abolish its ability to act as a calcium channel in artificial lipid bilayers (12). Following pre-incubation with N⁶-monobutyryladenosine 3′:5′ cyclic monophosphate (N⁶-mbcAMP) (0-1 mM) at 4° C. for 30 min, a membrane preparation was phosphorylated with γ[³²P] ATP at 37° C. for 5 min. Maximal inhibition of phosphorylation of annexin I by N⁶-mbcAMP occurred at 1 mM (FIG. 4).

In conclusion, we demonstrate novel Anx I phosphorylation and provide a route to understand the function(s) of the annexin gene family. We also provide a unifying hypothesis to explain the pleiotropic disorders in CF cell physiology by bringing together inflammation and secretion in a single pathway. When disordered histidine phosphorylation of Anx I is coupled to NDPK dysfunction in CF²⁹ (see Example 1), a mechanism for disease pathogenesis emerges.

Sample Preparation.

Swiss mice (wild-type) (6-10 weeks old) and mutant Cftr^{tm1Cam} mice¹² (4-12 weeks old) were used in this study. The mice were killed by exposure to CO₂. After exsanguination, tracheas were removed and cut open longitudinally. Epithelium was scraped and dislodged into homogenisation buffer¹⁵. Pooled (at least 10 tracheae/prep; 4 tracheae for each part of the transfection experiments) tracheal scrapings were manually homogenised for 1.5 min and the homogenate spun at 600×g for 5 min at 4° C. The post nuclear supernatant was respun at 100,000×g for 2 h at 4° C. The pellet was resuspended in homogenisation buffer and spun for 30 min at 16,000×g at 4° C. (repeated 3 times to remove contaminating cytosol). Use of lactate dehydrogenase (LDH) as marker of cytosol showed no cytosolic contamination in pellet¹⁵. Aliquots of cytosol and final membrane- pellet were stored in liquid nitrogen. The ovine sample preparation has been described previously¹⁵.

Phosphorylation, Western blotting and immunoprecipitation.

As described previously¹⁵. Phosphorylation and Electronic Autoradiography - Briefly, proteins were phosphorylated with either 37 kBq γ[³²P] ATP or GTP (final nucleotide concentration, 16 nM) in 10 mM MOPS pH 7.9 containing 5 mM DTT. The reaction was terminated by adding 5x Laemmli sample buffer (Laemmli (1970) Nature 227, 680-685) and the proteins separated by SDS-PAGE using 12.5% polyacrylamide gels. Pre-stained molecular weight markers were used to avoid staining and destaining of gels prior to imaging and quantification. The incorporation of ³²PO₄ into individual protein bands was detected using electronic autoradiography (Canberra-Packard Instant Imager).

Western blotting - Proteins (10 μg), separated by SDS-PAGE, were transferred to PVDF membrane (Millipore) by semi-dry electrophoretic transfer (Pharmacia) using 0.8 mA/cm² for 1 h with 20% methanol added to standard SDS-PAGE running buffer. Pre-stained markers were used to confirm transfer. The primary antibody (1:2000) was an affinity purified, rabbit polyclonal directed against a carboxy terminal peptide of annexin I (epitope corresponding to amino acids 324-342, Santa Cruz Biotechnology). Horseradish Peroxidase (HRP) conjugated anti-rabbit secondary antibody (Scottish Antibody Production Unit) and ECL detection (Amersham) were used to locate annexin I.

Immunoprecipitation - Membranes were re-suspended in 100 μl 10 mM MOPS pH 7.9 and phosphorylated with 67 nM γ[³²P] ATP/GDP (500 nM for 5 minutes at 37° C. The reaction was terminated with 50 mM EDTA followed by the addition of 9 volumes of immunoprecipitation buffer (10 mM Tris-HCl pH 7.4, 2 mM EDTA, 1 mM NaF, 1 mM DTT. 1% sodium deoxycholate, 1% NP-40, 0.3 μM aprotonin, 0.2 μM PMSF). The mixture was pre-cleared with protein G-Sepharose beads (30 min at 4° C.), centrifuged at 4° C. at 350 g for 5 min and the supernatant incubated with either a rabbit polyclonal (or mouse monoclonal antibody) to annexin I (1μg) for 1 h at 4° C. New beads were added and the mixture incubated overnight at 4° C. The incubation mixture was centrifuged at 350 g for 5 min and the pelleted beads washed in 1 ml RIPA buffer (50 mM Tris-HCl pH 7.4, 1 % NP-40, 0.5% sodium deoxycholate, 5 mM EDTA, 150 mM NaCl). This wash step was repeated three times and then 50 μl of 5x sample buffer containing 100 mM DTT was added to the pellet, left at room temperature for 30 minutes and finally, spun at 420 g for 5 minutes. A 20 μl aliquot was then run on 12.5% polyacrylamide gels for analysis by electronic autoradiography.

Purification and identification of Anx I:

Membranes from sheep tracheal epithelia were solubilised with 0.25% glucopyranoside in 10 mM Tris-HCl pH 7.4 (+cocktail of protease inhibitors¹⁴) on ice for 1 h and centrifuged at 100 000×g for 30 min. The supernatant (100 ml) was incubated at 37° C. for 5 min with ATP (50 nM)+GDP (500 nM) to enhance phosphorylation of the 37 kDa protein 20-fold¹⁵. The reaction was stopped by simultaneously adjusting the temperature to 4° C. and pH to 5.0 with buffer A (0.1 M acetic acid/NaOH pH 5.0, 0.5 M NaCl, 0.4 AEBSF, 0.1% glucopyranoside). The sample (500 ml, 434 mg) was applied to a Chelating Sepharose fast flow column (50 ml) charged with ferric chloride, pre-equilibrated with buffer A and then washed in the same buffer (15 column volumes/stable OD₂₈₀˜0.01). The column was eluted with buffer A containing 20 mM sodium biphosphate. The eluate (100 ml, 1.13 mg) was dialysed against buffer B (20 mM Tris-HCl, pH 8.5) followed by anion exchange chromatography on POROS HQ 20 pre-equilibrated with buffer B. More than 95% of protein (OD₂₈₀) was in the anion exchange column ‘flow through fraction’. SDS-PAGE analysis of this fraction (Coomassie staining) showed a major band of 37 kDa and a minor band of 70 kDa. Tryptic peptide digests (in-gel) from the purified 37, 70 kDa protein bands were obtained and sequence analysis conducted.

On, discovering that p37 was; identical to annexin I, we modified the purification procedure and included a Ca²⁺-chelating agent to extract membrane associated calcium-binding proteins. The membrane fraction was incubated with 10 mM Tris-HCl pH 7.4 containing 5 mM BAPTA for 30 min on ice. Following centrifugation, the supernatant was phosphorylated with cold ATP/GDP and applied to a chelating Sepharose Fe³⁻ column as described above.

Protein sequence analysis.

N-terminal sequencing and “in-gel” tryptic peptide digests of the purified proteins were conducted according to published procedures (Hayes et at (1989) Biochem. J. 264, 437-445.

HPLC phosphoamino acid analysis:

As described previously¹⁹. In order to determine the nature of the phosphate linkage on [³²-P]-annexin I, a membrane fraction was labelled with γ[³²P] GTP or γ[³²P] ATP for 5 min at 37° C. The reaction was terminated with 5x Laemmli sample buffer. Phosphorylated protein (10 μg) was then incubated at 30° C. for 10 min in reaction mixtures containing phosphorylation buffer alone, 0.1 N HCl (pH 1), 0.1 N NaOH (pH 13) or 0.8 M hydroxylamine-0.1 M acetate (pH 5.2). Phosphoramidates are stable under alkali conditions and labile under acidic conditions whereas acyl phosphates are labile under both extremes. In the presence of hydroxylamine, both linkages are labile at pH<5.5 (8). On the other hand, serine and threonine linkages are acid stable and base labile, whereas tyrosine is relatively stable under both acid and base.

The precise phosphorylated residue was determined by HPLC phosphoamino acid analysis as described previously (Wei & Matthews (1991) Meth. Enymol. 200, 388-414). Briefly, proteins were phosphorylated and blotted onto PVDF as described above. The phosphorylated bands were excised, digested under alkaline conditions and analysed, in the presence of phosphoamino acid standards, by anion exchange HPLC coupled to fluorescence detection. Fractions were collected and radioactivity detected by Cerenkov counting.

References for Example 2

1. Kerem, B. S. et al (1989) Identification of the cystic fibrosis gene: genetic analysis. Science 245, 1073-1080.

2. Spilmont, C. et al (1996) CFTR is involved in membrane endocytosis but not in fluid-phase and receptor-mediated endocytosis in human respiratory epithelial cells. Biochem. Biophys. Res. Comm. 227, 182-188.

3. Biwersi, J. Emans, N. & Verkmann, A. S. (1996) Cystic fibrosis transmembrane conductance regulator activation stimulates endosome fusion in vivo. Proc. Natl. Acad. Sci. USA 93, 12484-12489.

4. Meile, L. Cordella-Meile E. Xing M. Frizzell & Mukherjee A. B. (1997) Cystic fibrosis gene mutation (ΔF508) is associated with an intrinsic abnormality in Ca²⁺-induced arachidonic acid release by epithelial cells. DNA and Cell Biology 16, 749-759

5. Khan, T. Z. et al (1995) Early pulmonary inflammation in infants with CF. Am. J. Resp. Crit. Care Med. 151, 1075-1082.

6. Gerke V. & Moss S. E. (1997) Annexins and membrane dynamics. Biochemica et Biophysica Acta 1357, 129-154.

7. Chap, H. et al (1991) Une homologie frappante entre le CFTR et les annexines medecine/sciences 7, 8-9.

8. Donnelly, S. R. & Moss, S. E. (1997) Annexins in the secretory pathway. Cell. Mol. Life Sci. 53, 533-538.

9. Piretti, M. & Flower, R. J. (1993) Modulation of IL-1-induced neutrophil migration by dexamethasone and lipocortin 1. J. Immunol. 150, 992-999.

10. Ahluwalia, A. et al. The biology of annexin I. In: Annexins: Molecular Structure to Cellular Function (ed. Seaton, B.) p161-199 (Springer Verlag, Heidelberg, 1996).

11. Ernst, J. D. Annexin functions in phagocytic leukocytes. In: Annexins: Molecular Structure to Cellular Function (ed. Seaton, B.) p81-96 (Springer Verlag, Heidelberg, 1996).

12. Mollenhauer, J. Annexins (1997) Cell. Mol. Life Sci. 53, 506-507.

13. Ratcliff, R. et al (1993) Production of a severe cystic fibrosis mutation in mice by gene targeting. Nature Genet. 4, 35-40.

14. Treharne, K. J. Marshall, L. J. & Mehta, A. (1994) A novel chloride dependent, GTP-utilizing protein kinase in plasma membranes from human respiratory epithelium. Am. J. Phsiol. (Lung Cell. Mol. Physiol.) 267, L592-L601.

15. Muimo, R. Banner, S. Marshall, L. J. & Mehta, A. (1998) Nucleoside Diphosphate kinase and Cl⁻-sensitive protein phosphorylation in apical membranes from ovine airway epithelium. Am. J. Respir Cell. Mol. Biol. 18, 270-78.

16. Marshall, L. J. Muimo, R. Riemen, C. E. & Mehta, A. (1999) Na⁺ and K⁺ regulate the phosphorylation state of nucleoside diphosphate kinase in human airway epithelium. Am. J. Phys. Cell. 276, C109-119.

17. Andersson, B. and Porath, J. (1986) Isolation of phosphoproteins by immobilized metal affinity (Fe³+) affinity chromatography.

Anal. Biochem. 154, 250-254.

18. Rothhut, B. (1997) Participation of annexins in protein phosphorylation. Cell. Mol. Life Sci. 53, 522-526.

19. Wei, Y. and Matthews, H. R. (1991) Identification of phosphohistidine in proteins and purification of protein histidine kinases. Meth. Enzymol. 200, 388-414.

20. Cohen, B. E. et al (1995) Cyclic 3′-5′-adenosine monophosphate binds to annexin I and regulates calcium-dependent membrane aggregation and ion channel activity. Febs. Lett. 377, 444-450.

21. Weng et al (1993) Crystal structure of human annexin I at 2.5 Å resolution. Protein Sci. 2, 448-458.

22. Boucher, R. C. et al (1988) Evidence for reduced Cl and increased Na permeability in cystic fibrosis human primary cell cultures. J Pkysiol. (London) 405, 77-103.

23. Schwiebert, E. M. et at (1994) Heterotrimeric G proteins, vesicle trafficking and CFTR Cl⁻ channels. Am. J. Physiol. 267, C272-281.

24. Croxtall, J. D. Choudhury, Q. Newman, S. Flower, R. J. (1996) Lipocortin I and the control of cPLA2 activity in A549 cells. Biochem Pharmacol. 52, 351-356.

25. Strandvik, B. et at (1988) Relation between defective regulation of arachidonic acid release and symptoms in cystic fibrosis. Scand. J. Gastroenterol. 23 (suppl. 143), 1-4.

26. Kaufman, M. Leto, T. & Levy, R. (1996) Translocation of annexin I to plasma membranes and phagosomes in human neutrophils upon stimulation with opsonised zymosan: possible role in phagosome function. Biochem. J. 316, 35-42.

27. Dorin, J. R. et at (1987) A clue to the basic defect in cystic fibrosis from cloning the CF antigen gene. Nature 326, 614-617.

28. Goddard, C. A. et at (1997) A second dose of a CFTR cDNA-liposome complex is as effective as the first dose in restoring cAMP dependent chloride secretion to null CF mice trachea. Gene Therapy 4, 1231-1236.

29. Example 1.

### Example 3

Collection of nasal epithelial cells; their fractionation and determination of phosphorylation of membrane proteins

Collection of nasal respiratory epithelial cells

Immediately after anesthesia, respiratory epithelial cells are brushed from the inferior nasal turbinate epithelium with a cytology brush and dislodged into a nutrient medium (medium 199, Flow). The cells are separated from contaminating inflammatory cells and mucus by repeated cycles of sedimentation (1 g for 10 min at 4° C.) followed by replacement of the supernatant with fresh medium. Staining of the supernatant and cell pellets with hematoxylin and eosin showed that contaminating inflammatory cells constituted<10% of the cells in the initial pellet and that this ratio was reversed in the supernatant. Further wash cycles reduced the containing to<1%.

Fractionation of cells

The washed cell pellets (⁻3×10⁶ cells/pellet) were resuspended twice in ice-cold phosphate-buffered saline and centrifuged at 150 g for 5 min at 4° C.; the final cell pellet was resuspended in 1.5 ml of ice-cold homogenization buffer [6% sucrose (wt/vol) containing freshly prepared protease inhibitors; see below]. The cells were sonicated twice at 15 μm (Soniprep, MSE) for 10 s, with care taken to cool the microprobe tip in melting ice for 10 min before sonication and for 1 min between each sonication step. The sonicated cells (homogenate) were spun at 6,000 revolution/min (rpm) (2,900 g for 5 min at 4° C.) to remove cellular debris and unbroken cells (6K pellet). The supernatant (2 ml) was then loaded onto a discontinuous sucrose gradient (1 ml each of 20, 30, and 65% (wt/vol) sucrose] in a precooled 5-ml polyallomer tube (Kontron) and spun at 150,000 g (50,000 rpm in a precooled Kontron TST 55.5 swing-out rotor) for 60 min at 2° C. The gradient was unloaded from above, the upper 2 ml of cytosol were separated, and three separate sets of membranes were preserved: the 20-30% and 30-55% interfaces and the pellet at the bottom of the 5-ml tube below the 55% sucrose. The two sets of membranes above the 55% interface were each repelleted at 14,000 rpm (15,800 g) for 5 min at 4° C. Each pellet was resuspended in the appropriate buffer for the marker assays (FIG. 1) or in ice-cold 10 mM 3-(N-morpholino) propanesulfonic acid (MOPS, pH 7.9 with KOH, final concentration of K 101 mM) containing 0.05% Triton X-100, 20 μM dithiothreitol, and 0.5% dimethyl sulfoxide (DMSO) for the phosphorylation experiments. Protein concentrations were determined at each fractionation stage by use of a commercial reagent (Biorad), and the volume of MOPS membrane buffer added to the final pellet was adjusted to yield a protein concentration of 600 μg/ml. The membranes were used immediately or aliquoted and stored in liquid nitrogen.

All sucrose solutions contained the following protease inhibitors: 1 mM phenylemethylsulfonyl fluoride, leupeptin (10 μg/ml), and pepstatin (10 μg/ml); 1 μg/ml); 1 μl of 1 M disopropyl fluorophosphate was added to each cell pellet immediately before sonication.

Phosphorylation of membranes with [γ-³²P]ATP or [γ-³⁵S]ATP

Aliquots (15 μg) of membranes (resuspended in 25 μl of MOPS membrane buffer) were incubated with 37 kBq of [γ-³²P]GTP/ATP (1:10 dilution in 10 mM MOPS of a stock solution of 370 MBq/ml, final concentration 16 nM) or [γ-⁼S]GTP/ATP (1 μl of undiluted label, 462.5 MBq/ml, final concentration 372 nM at 37° C. Time course analysis showed that maximal phosphorylation was achieved within 1 min, and no increase was seen between 1 and 5 min. A standard time of 5 min was used in all experiments. In all experiments, 1 μl of labelled nucleotide was spotted onto the side of the tube and the reaction was started by a rapid spin to mix reagents. The N-methyl-D-glucamine (NMDG) salts were made by titration with the appropriate acid, and control experiments showed that phosphorylation was unaffected by changing the pH between 7.6 and 7.9 and that 10 mM MOPS had sufficient buffering capacity to prevent pH changes within this range.

Quantification of phosphorylation

Phosphorylation was terminated after 5 min by addition of 5 x Laemmli sample buffer at 37° C. followed by rapid mixing. Proteins were separated by sodium dodecyl sulfate polyacrylamide gel electrophoresis with 10% gels on a Bio-Rad Protean II slab cell. The gels were stained with Coomassie Blue R, destained, dried, and (for qualitative analysis) autoradiographed against preflashed Hyperfilm MP (Amersham International) at −70° C. The incorporation of ³²PO₄ and ³⁵S-PO₃ into individual protein bands was quantified with a Canberra-Packard Instant Imager.

1. A method of determining whether a patient has, or is responding to treatment for, cystic fibrosis the method comprising the steps of (1) obtaining a suitable epithelial cell sample from the patient, (2) determining whether nucleotide diphosphate kinase (NDPK) function or state is altered compared to its function or state in a control epithelial cell.

2. A method according to claim 1 wherein phosphorylation of NDPK is altered.

3. A method according to claim 1 wherein nucleoside triphosphate production from a given nucleoside diphosphate is measured.

4. A method of determining whether a patient has, or is responding to, treatment for, cystic fibrosis the method comprising the steps of (1) obtaining a suitable epithelial cell sample from the patient, (2) determining whether histidine phosphorylation of annexin is altered compared to its phosphorylation in a control epithelial cell.

5. A method according to claim 4 wherein the histidine is His246 or His293 of annexin.

6. A method according to any one of claims 1 to 5 wherein the epithelial cell sample from the patient is a lung cell sample or a nasal cell sample.

7. A method of classifying a disease state associated with epithelial cell dysfunction in a patient, the method comprising (1) obtaining a suitable epithelial cell sample from the patient and (2) determining for one or more of the following whether the measured parameter is altered compared to a control epithelial cell the measured parameters being: (i) nucleoside diphosphate kinase (NDPK) function, (ii) phosphorylation of annexin, (iii) phosphorylation of other membrane proteins, and (iv) ATPase activity.

8. A method according to claim 7 wherein in step (ii) phosphorylation of annexin at His246 or His293 is measured.

9. A method according to claim 7 wherein each of parameters (i) and (ii) are measured in the sample from the patient and compared to the control sample.

10. A method according to claim 7 wherein each of parameters (i), (ii) and (iii) are measured in the sample from the patient and compared to the control sample.

11. A method according to claim 7 wherein all of parameters (i) to (iv) are measured in the sample from the patient and compared to the control sample.

12. A method according to any one of claims 7 to 11 wherein the epithelial cell sample from the patient is a lung, cell sample or a nasal cell sample.

13. A method according any one of claims 7 to 12 wherein the effectiveness of a treatment for cystic fibrosis is being tested on the patient.

14. A method of identifying a compound useful in treating cystic fibrosis or which may aid the identification of a compound useful in treating cystic fibrosis the method comprising identifying a compound which modulates or restores nucleoside diphosphate kinase activity.

15. A method according to claim 14 wherein phosphorylation of NDPK is altered.

16. A method according to claim 14 wherein nucleoside triphosphate production from a given nucleoside diphosphate is altered.

17. A method of identifying a compound useful in treating cystic fibrosis or which may aid the identification of a compound useful in treating cystic fibrosis the method comprising identifying a compound which modulates histidine phosphorylation of annexin.

18. A method according to claim 17 wherein the histidine phosphorylation of annexin is at His246 or His293.

19. A method of identifying a compound useful in treating cystic fibrosis or which may aid the identification of a compound useful in treating cystic fibrosis the method comprising identifying a compound which modulates the interaction between any of cystic fibrosis transmembrane conductance regulator protein (CFTR), nucleoside diphosphate kinase (NDPK) and annex.

20. A method according to any one of claims 14 to 19 wherein the method is carried out in vivo.

21. A method of identifying a compound useful in treating cystic fibrosis or which may aid identification of a compound useful in treating cystic fibrosis the method comprising identifying a compound which substantially changes one or more of the following parameters from the state found in a cystic fibrosis epithelial cell to the state found in a normal cell, namely (i) nucleoside diphosphate kinase (NDPK) function, (ii) phosphorylation of annexin, (iii) phosphorylation of other membrane proteins such as p11 and p116, and (iv) ATPase activity.

22. A method according to claim 21 wherein the histidine phosphorylation of annexin is at His246 or His293.

23. A compound identified by the method of any one of claims 14 to 22.

24. A compound according to claim 23 for use in medicine.

25. A method of treating CF the method comprising administering to a patient a compound which modulates nucleoside diphosphate kinase activity or a compound which modulates histidine phosphorylation of annexin or a compound which modulates the interaction between any of cystic fibrosis transmembrane conductance regulator protein (CFTR), nucleoside diphosphate kinase (NDPK) and annexin.

26. A method according to claim 25 wherein the histidine phosphorylation of annexin is at His246 or His293.

27. Use of a compound as defined in claim 36 in the manufacture of a medicament for treating cystic fibrosis.

28. A peptide of relative molecular mass less than 6500 comprising at least ten consecutive amino acid residues surrounding the phenylalanine 508, or at least ten consecutive residues including a portion of the region between residues 508 and 551, in the polypeptide sequence of human cystic fibrosis transmembrane regulator (CFTR), or a variant or precursor thereof.

29. A peptide according to claim 28 having between 12 and 50 amino acid residues.

30. A peptide according to claim 29 having between 12 and 30 amino acid residues.

31. A peptide according to claim 30 having between 12 and 20 amino acid residues.

32. A peptide according to any one of claims 28 to 31 which has the sequence KENIIFGVSYDEYR.

33. A peptide according to any one of claims 28 to 32 further comprising a lipid-solubilising moiety.

34. A peptide according to claim 33 wherein the lipid-solubilising moiety is a lipid.

35. A peptide according to claim 33 wherein the lipid-solubilising moiety is a cholesterol.

36. A peptide according to claims 33 or 34 wherein the lipid-solubilising moiety is a fatty acid.

37. A peptide according to claim 36 where in the fatty acid is any one of palmitic or myristic acid.

38. A peptide according to any one of claims 28 to 37 for use in medicine.

39. A pharmaceutical formulation comprising a peptide according to any one of claims 28 to 37 and a pharmaceutically acceptable carrier.

40. A method of treating cystic fibrosis or a chronic sputum producing disorder the method comprising administering to the patient an effective amount of a peptide according to any one of claims 28 to 37.

41. A method according to claim 40 wherein the peptide is administered in a nebulised form.

42. Use of a peptide according to any one of claims 28 to 37 in the manufacture of a medicament for treating cystic fibrosis or a chronic sputum producing disorder.

43. A peptide of relative molecular mass less than 6500 comprising at least five consecutive residues surrounding histidine 246 of annexin.

44. A peptide of relative molecular mass less than 6500 comprising at least five consecutive residues surrounding histidine 293 of annexin.

45. A peptide according to claim 43 or 44 wherein the said histidine residue is phosphorylated.

46. A method of raising an antibody reactive with histidine phosphorylated annexin, the method comprising using a peptide according to claim 45 as an immunogen.

47. A method according to claim 46 wherein the said peptide is combined with a carrier or adjuvant or both.

48. An antibody obtainable by the method of claim 46 or 47.

49. An antibody reactive against annexin phosphorylated at histidine 246 but not reactive against annexin not phosphorylated at histidine 246.

50. An antibody reactive against annexin phosphorylated at histidine 293 but not reactive against annexin not phosphorylated at histidine 293.
